(19) United States
(12) Patent Application Publication
(10) Pub. No.: US 2010/0280104 A1
(43) Pub. Date: Nov. 4, 2010

(54) METHODS AND KITS FOR DIAGNOSIS AND TREATMENT OF CELL-CELL JUNCTION RELATED DISORDERS

(75) Inventors: Christopher A. Walsh, Chestnut Hill, MA (US); Yuanyi Feng, Chicago, IL (US); Volney Sheen, Wellesley, MA (US)

Correspondence Address: CLARK & ELBING LLP, 101 FEDERAL STREET, BOSTON, MA, 02110, US

(73) Assignee: BETH ISRAEL DEACONESS MEDICAL CENTER, Boston, MA (US)

(21) Appl. No.: 12/517,140

(22) PCT Filed: Dec. 3, 2007
(86) PCT No.: PCT/US2007/024754

§ 371 Date: Jul. 7, 2010

Related U.S. Application Data
(60) Provisional application No. 60/872,169, filed on Dec. 1, 2006.
(60) Provisional application No. 60/872,731, filed on Dec. 4, 2006.

(51) Int. Cl. A61K 31/7088; G01N 33/68; A61P 9/10; A61L 29/16

(52) U.S. Cl. 514/44 R; 435/7.21; 436/86; 623/1.42

(57) ABSTRACT

We have discovered that loss of Filamin A function results in weakened cell-cell junctions and vascular defects in genetically engineered mice. In addition, we have shown that patients with mutations in the gene coding for Filamin A exhibit high rates of cardiovascular disorders. On the basis of this discovery, the present invention features methods and kits for diagnosis of cell-cell junction related disorders (e.g., atherosclerosis, aortic aneurysm, or any disease described herein), as well as screening methods

### STATEMENT AS TO FEDERALLY FUNDED RESEARCH

The United States Government has a paid-up license in this invention and the right in limited circumstances to require the patent owner to license others on reasonable terms as provided for by the terms of P01NS40043 and 2R37 35129, each awarded by National Institute of Neurological Disorders and Stroke, and K01MH065338, awarded by the National Institute of Mental Health.

### BACKGROUND OF THE INVENTION

The invention relates to use of Filamin A in the diagnosis and treatment of cell-cell junction related disorders such as atherosclerosis or any disease described herein.

Filamin A (FLNA) is a widely studied actin binding protein. FLNA was first identified by its ability to crosslink actin filaments and since has been shown to bind more than 30 proteins with diverse functions.

Human FLNA loss of function mutations cause periventricular heterotopia (PH), in which abnormally migrated neurons are present near the lateral ventricle, deep beneath their normal locations. PH is an X-linked, male-lethal disease in which affected females have seizures. Because a FLNA-deficient cell line shows severe motility defects, PH has been interpreted as reflecting arrest of neuronal migration during cerebral cortical development. Subsequently, specific FLNA missense alleles were associated with skeletal dysplasias, including otopalatodigital (OPD) syndrome types 1 and 2, Frontometaphyseal dysplasia (FMD) and Melnick-Needles syndrome (MES), though the effects of these missense alleles on FLNA function remained elusive.

Thus, prior to the present invention, other effects of loss of FLNA function were not well understood.

### SUMMARY OF THE INVENTION

We have identified a role for Filamin A in the maintenance of cell-cell junctions in endothelial cells forming blood vessels and have shown that loss of Filamin A function results in weakness of these junctions and endothelial cell rupture. On this basis, the invention features methods and kits for diagnosis of cell-cell junction related disorders and screening and treatment methods for such disorders (e.g., atherosclerosis or any condition described herein including aortic aneurysm).

Accordingly in one aspect, the invention features a method for diagnosing a cell-cell junction related disorder or an increased propensity thereto in a patient (e.g., an adult, a human, or an adult human). The method including obtaining a biological sample (e.g., a blood sample) from the patient and determining the level of Filamin A in the sample, where an alteration in the level of Filamin A is diagnostic of a cell-cell junction related disorder (e.g., any disorder described herein) or an increased propensity thereto.

In another aspect, the invention features a second method for diagnosing a cell-cell junction related disorder or an increased propensity thereto in a patient (e.g., an adult, a human, or an adult human). The method includes obtaining a first biological sample (e.g., a blood sample) from the patient, determining the level of Filamin A in the first sample, obtaining a second biological sample from the patient within five years (e.g., within four, three, two, or one years, or within 9, 6, 3, 2, or 1 months) of the first sample, and determining the level of Filamin A in the second sample, where a change in the level in the second sample as compared to the first sample is diagnostic of a cell-cell junction related disorder (e.g., any disorder described herein) or an increased propensity thereto. Third, fourth, fifth, sixth, seventh, or more samples may further be obtained in a similar manner, and comparisons between any of the various samples may be made to diagnose a cell-cell junction related disorder or an increased propensity thereto.

In either of the above two aspects, the cell-cell junction related disorder may be atherosclerosis. The determining step or steps may be performed in conjunction measuring the level of a vascular endothelial cell marker (e.g., PECAM (CD31) or factor VIII).

In another aspect, the invention features a method for diagnosing a cell-cell junction related disorder (e.g., any disorder described herein) or an increased propensity thereto in a patient. The method includes obtaining a biological sample (e.g., a blood sample) from the patient (e.g., an adult, a human, or an adult human), and (b) determining whether the sample includes (i) mutant Filamin A, (ii) a polynucleotide encoding a mutant Filamin A, or (iii) a polynucleotide that encodes Filamin A and includes a polymorphism, where the detection of (i), (ii), or (iii) is diagnostic of a cell-cell junction related disorder (e.g., atherosclerosis) or an increased propensity thereto. The mutant or polymorphism may be the result of a somatic mutation in the Filamin A gene. The somatic mutation may be in a vascular endothelial cell.

In another aspect, the invention features a kit including an antibody specific for Filamin A and instructions for use in diagnosing a cell-cell junction related disorder (e.g., atherosclerosis or any disorder described herein) or a propensity thereto.

In another aspect, the invention features a kit including an antibody specific for Filamin A bound to a solid support, a first soluble antibody specific for Filamin A, and instructions for use. The kit may further include a second soluble antibody with a detectable label, where the second antibody is specific for the first soluble antibody.

In another aspect, the invention features a method for identifying a candidate compound useful for treating a cell-cell junction related disorder (e.g., atherosclerosis or any disorder described herein) in a patient. The method includes contacting a Filamin A protein (e.g., a human Filamin A protein) with a compound (e.g., selected from a chemical library), and measuring the activity (e.g., actin binding activity) of the Filamin A, where an alteration in Filamin A activity in the presence of the compound relative to activity in the absence of the compound identifies the compound as a candidate compound for treating a cell-cell junction related disorder in the patient. The Filamin A protein may be in a cell. The method may be performed in vitro.

In another aspect, the invention features a method for identifying a candidate compound useful for treating a cell-cell junction related disorder (e.g., atherosclerosis or any disorder described herein) in a patient. The method includes contacting a Filamin A protein (e.g., a human Filamin A protein) with a compound (e.g., selected from a chemical library), and measuring the binding of the compound to the Filamin A protein, where specific binding of the compound to the Filamin A protein identifies the compound as a candidate compound for treating a cell-cell junction related disorder in the patient. The method may be performed in vitro.

In another aspect, the invention features a method for treating a cell-cell junction related disorder (e.g., atherosclerosis or any disorder described herein) in a patient (e.g., an adult, a human, or an adult human) in need thereof. The method includes administering a composition that increases the expression or activity of Filamin A in the patient in an amount sufficient to treat the patient. The increase in expression or activity may take place in an endothelial cell (e.g., a vascular endothelial cell). The composition may include Filamin A protein or a polynucleotide encoding Filamin A (e.g., where the polynucleotide is part of a vector or is coated onto a stent).

By “Filamin A” is meant a polypeptide with at least 50%, 60%, 70%, 80%, 90%, 95%, or 99% sequence identity to SEQ ID NO:1, or a fragment thereof (FIG. 8) or a polypeptide encoded by a polynucleotide that hybridizes to a polynucleotide encoding SEQ ID NO:1, or a fragment thereof.

By “fragment” is meant at least 4, 5, 10, 20, 50, 100, 250, or 500 nucleic or amino acids. Exemplary fragments include C-terminal truncations, N-terminal truncations, or truncations of both C- and N-terminals.

By “patient” is meant either a human or non-human animal.

“Treating” a disease or condition in a subject or “treating” a patient having a disease or condition refers to subjecting the individual to a pharmaceutical treatment, e.g., the administration of a drug, such that at least one symptom of the disease or condition is decreased, stabilized, or prevented.

By “specifically binds” or “specific binding” is meant a compound or antibody which recognizes and binds a desired polypeptide but which does not substantially recognize and bind other molecules in a sample, for example, a biological sample, which naturally includes a polypeptide of the invention.

By a “decrease” in the level of expression or activity of a gene is meant a reduction in protein or nucleic acid level or activity in a cell, a cell extract, or a cell supernatant. For example, such a decrease may be due to reduced RNA stability, transcription, or translation, increased protein degradation, or RNA interference. Preferably, this decrease is at least 5%, 10%, 25%, 50%, 75%, 80%, or even 90% of the level of expression or activity under control conditions.

By an “increase” in the expression or activity of a gene or protein is meant a positive change in protein or nucleic acid level or activity in a cell, a cell extract, or a cell supernatant. For example, such a increase may be due to increased RNA stability, transcription, or translation, or decreased protein degradation. Preferably, this increase is at least 5%, 10%, 25%, 50%, 75%, 80%, 100%, 200%, or even 500% or more over the level of expression or activity under control conditions.

An “alteration” may be an increase or a decrease.

By “biological sample” or “sample” is meant a sample obtained from an organism or from components (e.g., cells) of an organism. The sample may be of any biological tissue or fluid. Frequently the sample will be a “clinical sample” which is a sample derived from a subject. Such samples include, but are not limited to, sputum, blood, blood cells (e.g., white cells), tissue or fine needle biopsy samples, urine, peritoneal fluid, and pleural fluid, or cells. Biological samples may also include sections of tissues such as frozen sections taken for histological purposes.

By “vascular endothelial cell marker” is meant any protein that is expressed in vascular endothelial cells, but not substantially expressed in another type of cell. A marker may be expressed at least 1.2, 1.5, 2, 5, 10, 25, or 100-fold more in a vascular endothelial cell as compared to another type of cell.

By “cell-cell junction related disorder” is meant any disease, condition, or disorder that is associated with dysfunction of or defects in cell-cell junctions such as adherens junctions (AJs). Diseases associated with dysfunction of cell-cell junctions include vascular diseases such as atherosclerosis, aortic aneurysm, patent ductus arteriosus, or valvular heart disease. Other cell-cell junction related disorders include epithelial cancers such as solid tumors (e.g., lung, breast, prostate, colon, and bladder cancer). In cancer, adherens junctions may negatively regulate cancer progression and carcinogenesis. β-Catenin, which also localizes to adherens junctions, is activated in many tumors because of loss of its localization to adherens junctions. β-Catenin is an important oncogene, and we believe there is a connection between Filamin A and catenins, as they co-localize and both bind to actin. Cancer metastasis of other tumors may also be mediated by adherens junctions, as metastases can enter distal sites by passing through the adherens junctions of vascular endothelial cells.

By “adult” is meant a mature organism, such as a human. Adult humans are at least 18, 20, 21, 25, 30, 40, 50, 60, or 70 years of age.

By “somatic mutation” is meant a spontaneously arising sequence change in a cell of an organism. Somatic mutations can result in changes in the coding sequence of a gene and may therefore affect the function of the protein. Alternatively, a somatic mutation can arise in a regulatory region of a gene (e.g., promoter, enhancer, suppressor, region) and can alter expression of the gene.

By a “compound,” “candidate compound,” or “factor” is meant a chemical, be it naturally-occurring or artificially-derived. Compounds may include, for example, peptides, polypeptides, synthetic organic molecules, naturally-occurring organic molecules, nucleic acid molecules, and components or combinations thereof

By “stent” is meant a slender thread, rod, or catheter lying within the lumen of a vessel used to provide support and to assure patency of an intact but contracted lumen.

Other features and advantages of the invention will be apparent from the following Detailed Description, the drawings, and the claims.

### BRIEF DESCRIPTION OF THE DRAWINGS

FIGS. 1A-1D show the targeting strategy for Flna conditional knockout and null mouse. FIG. 1A shows a Neo-TK cassette flanked by loxP sites was inserted downstream of a 2.3 kb fragment containing exon 2 and intron 2; and upstream of an 8 kb fragment containing exons 3-11 of mouse Flna. A third loxP site was inserted into intron 7. The targeting vector was transfected into embryonic stem cells to generate Flna targeted clones (Step I). The Flna recombinant ES cells were further transfected with a Cre recombinase expressing plasmid; selected for ganciclovir resistance (the loss of TK expression); and screened for deletion of the Neo-TK cassette alone (Step II), or with exon 3 through 7 of Flna (Step III). FIG. 1B shows Southern analysis of Flna gene targeted (FlnAT), FlnA knockout (KO) and conditional knockout (CKO, or floxed) ES cells. Genomic DNA was digested with Kpn I. The probe (P) used for Southern blotting is denoted as P in A. The initial Flna targeted allele showed an 8.4 kb band instead of the 9.7 kb band in the wild type allele. After Cre recombination, the 8.4 kb band reduced to 7.5 kb in the Flna knockout (KO) allele and to 9.7 kb in the conditional (floxed or CKO) allele. FIG. 1C shows Northern analysis of Flna null and conditional null ES cells. Blot was hybridized with probes from the N-terminal and C-terminal portions of Flna cDNA as well as a probe to the Flnb cDNA. Flna KO cells expressed a single Flna message with the deletion of exons 3 through 7, but showed no up-regulation of Flnb. FIG. 1D shows an immunoblot of Flna mutant ES cells shows the complete deletion of Flna protein in Flna knockout allele (KO), and unaltered Flna protein level in Flna floxed allele (conditional knockout or CKO). The blot was probed with antisera against peptides from both the mouse Flna amino terminal (anti-FLNA-N), and carboxyl terminal (anti-FLNA-C) sequences. The blot was also probed with an antibody to Flnb, which showed that its level of expression is not increased in the absence of Flna. α-tubulin (Tub) was used as a loading control.

FIG. 2A-2D show vascular defects in Flna knockout embryos. FIG. 2A shows a dying E14 Flna null mutant embryo and its wild type littermate.

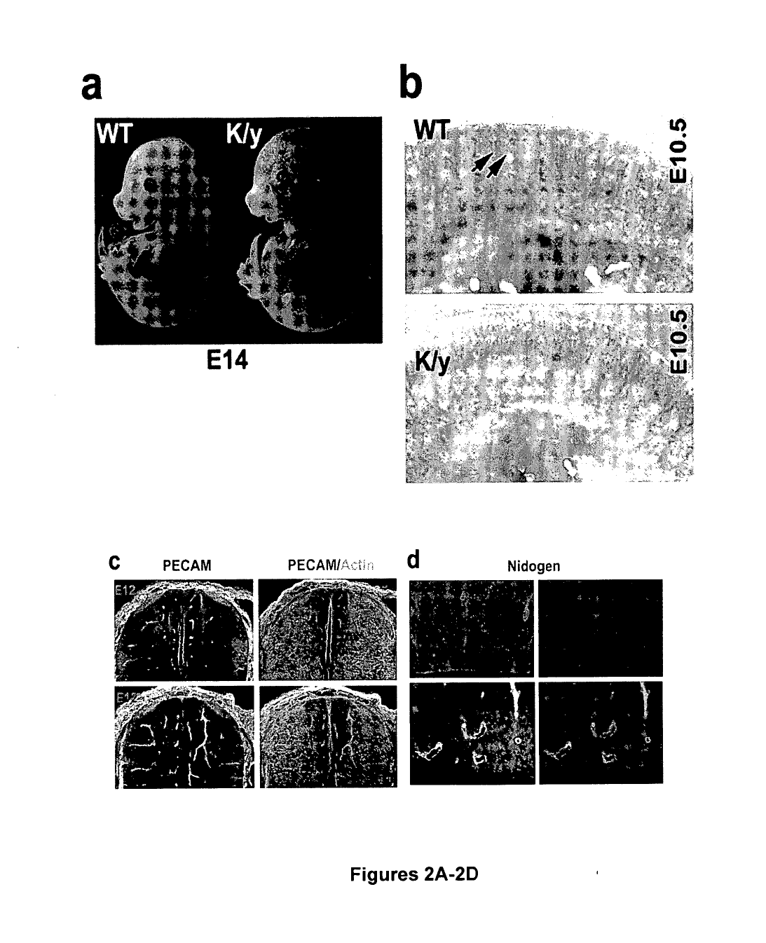

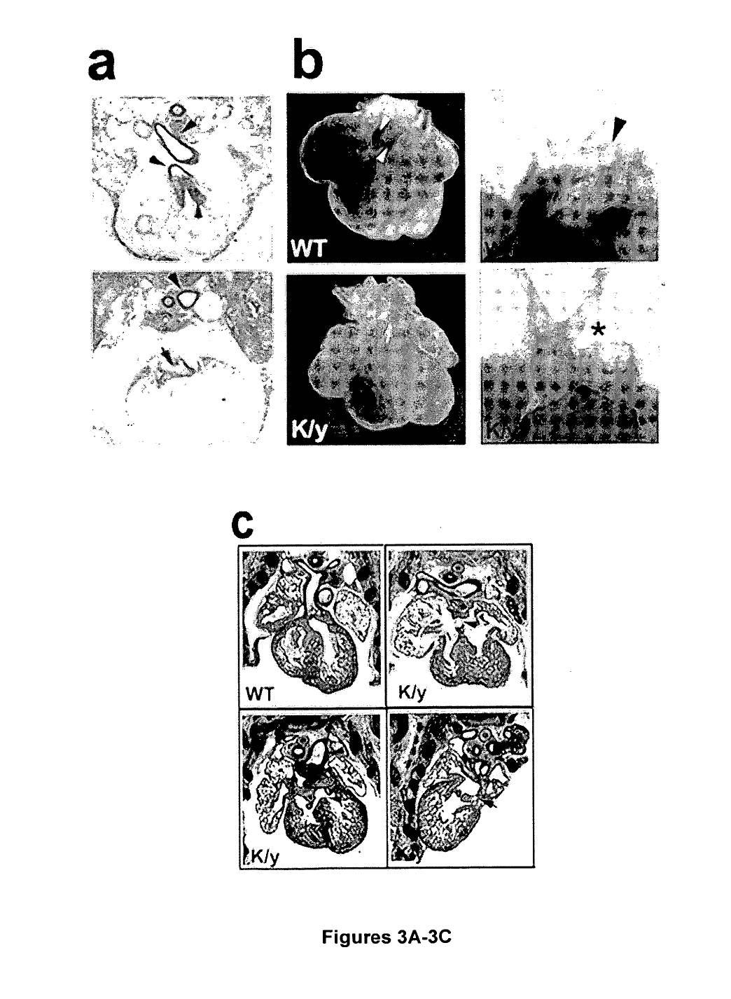

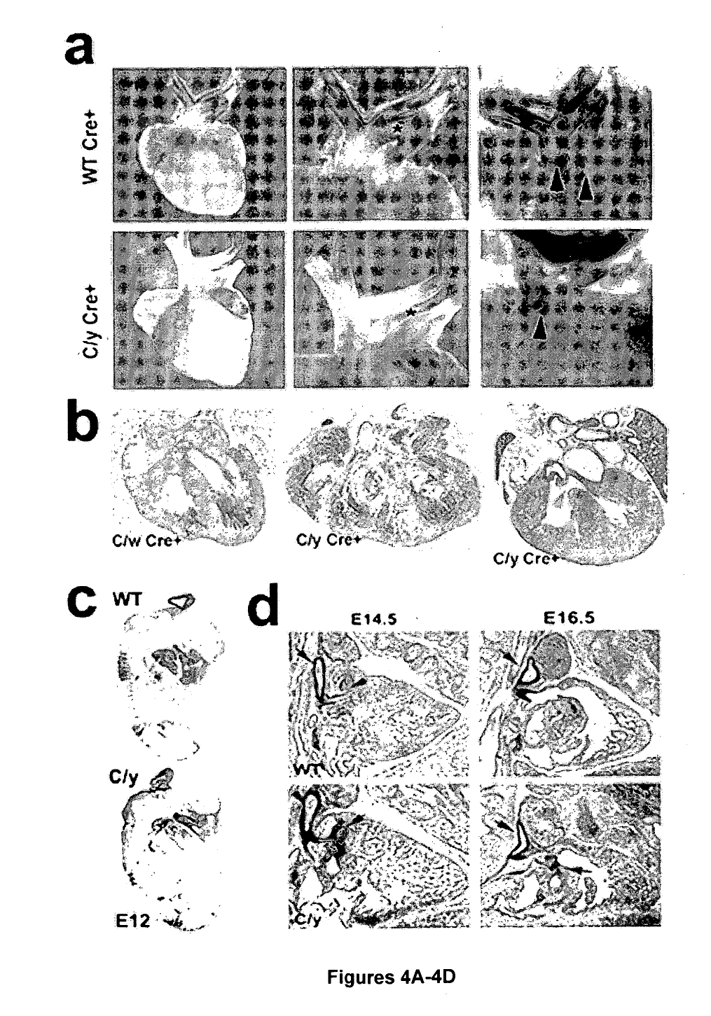

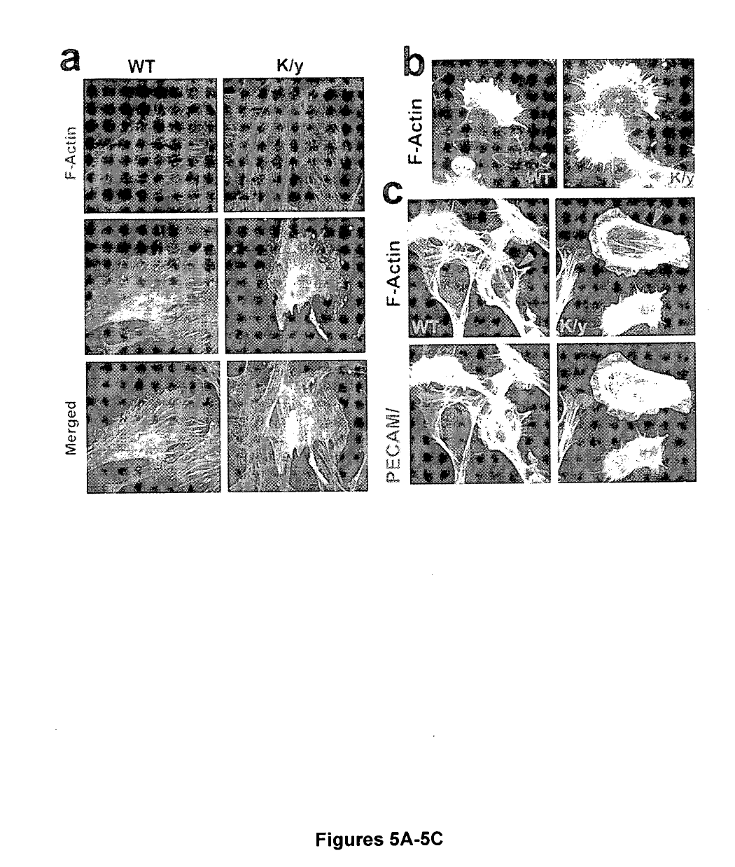

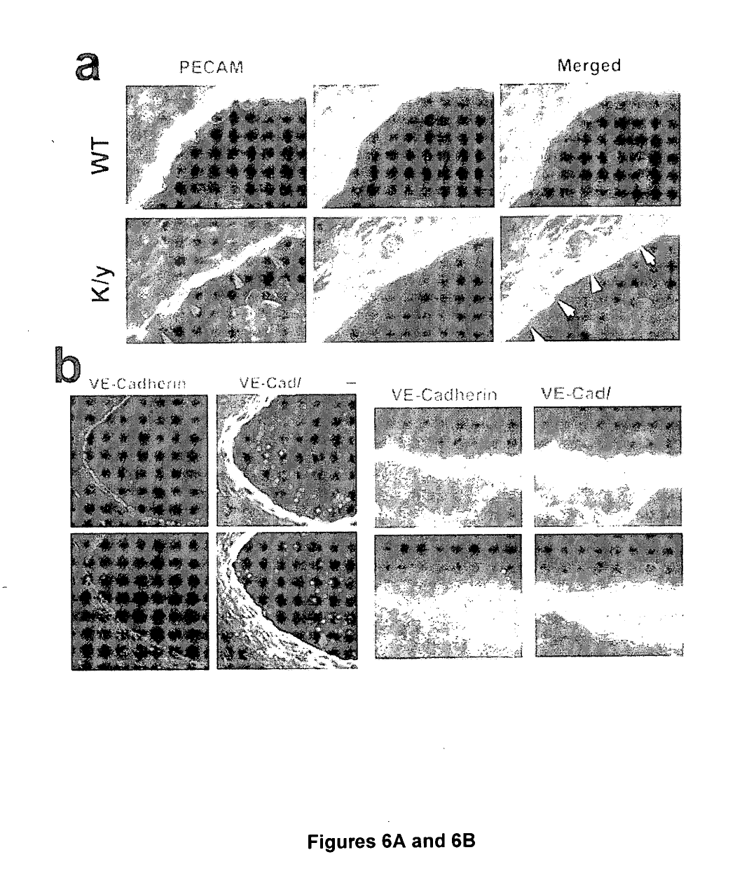

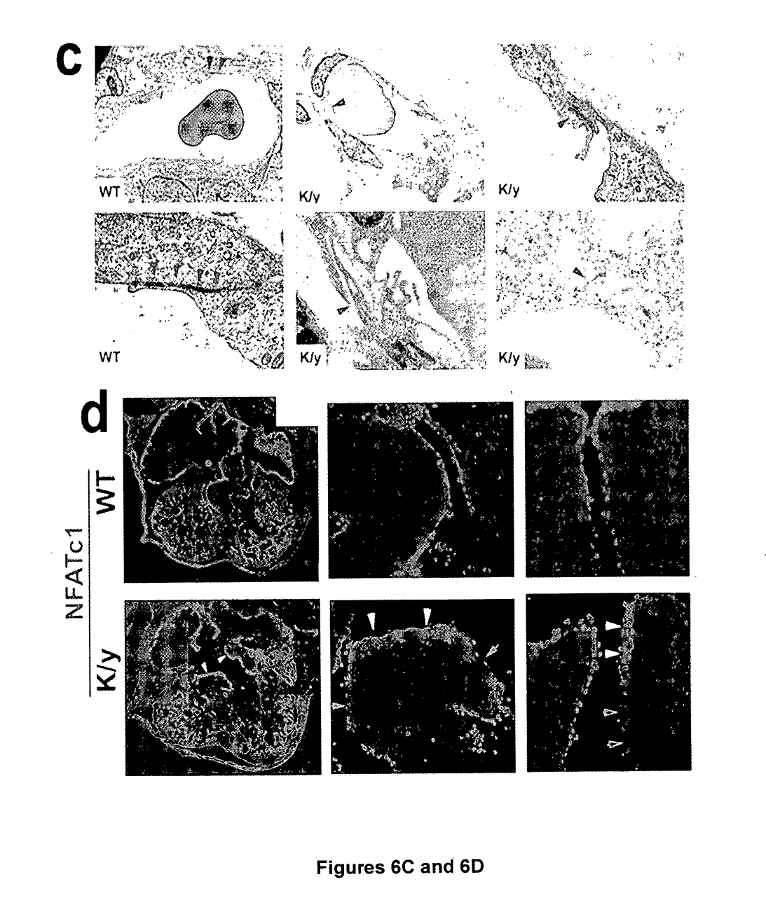

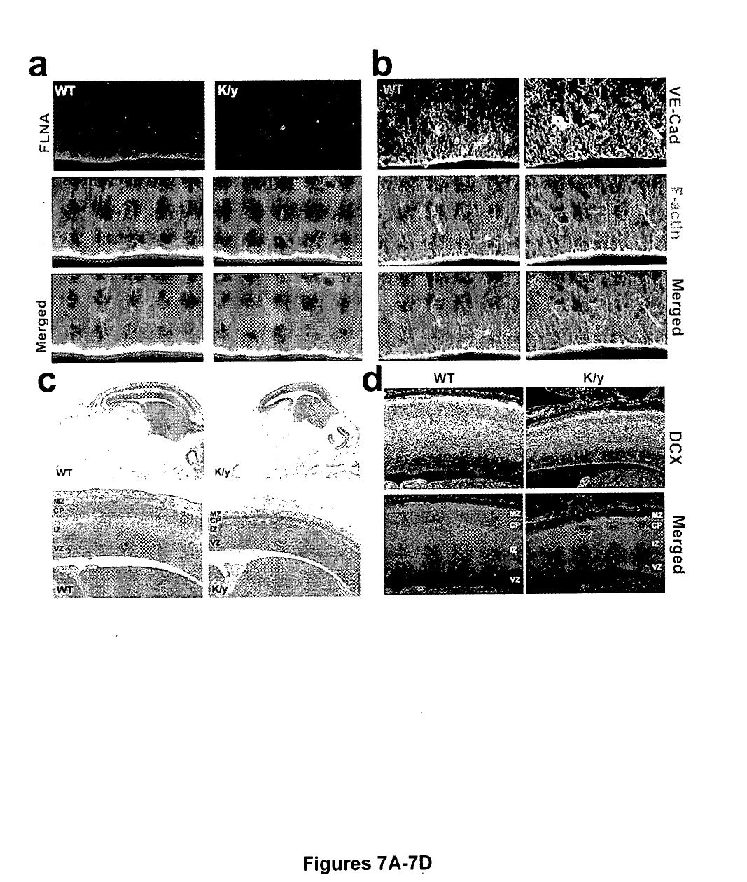

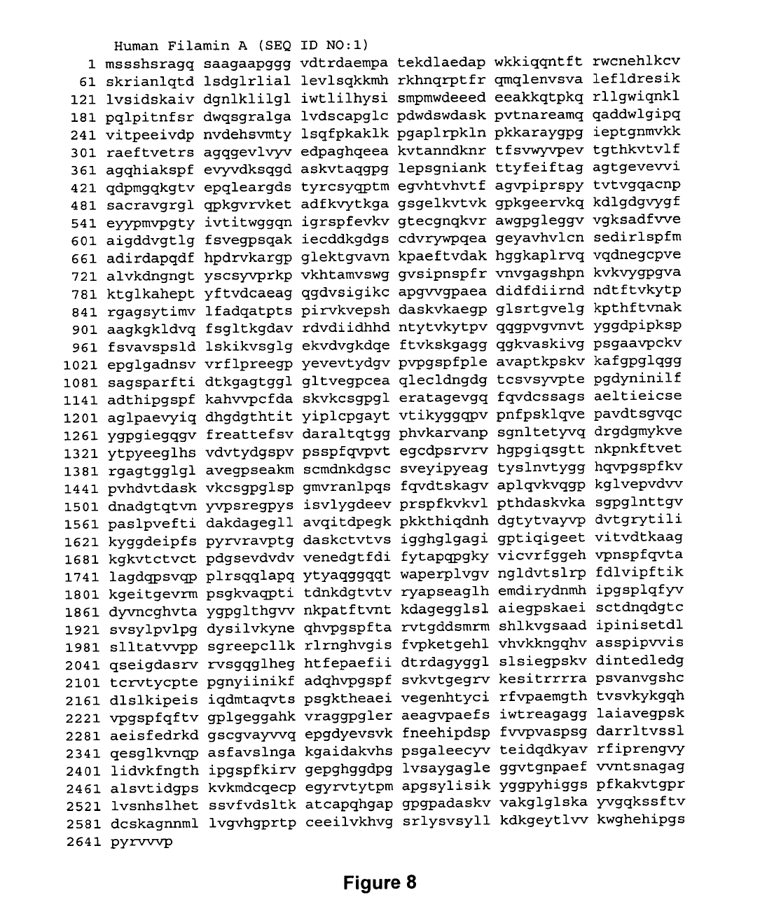

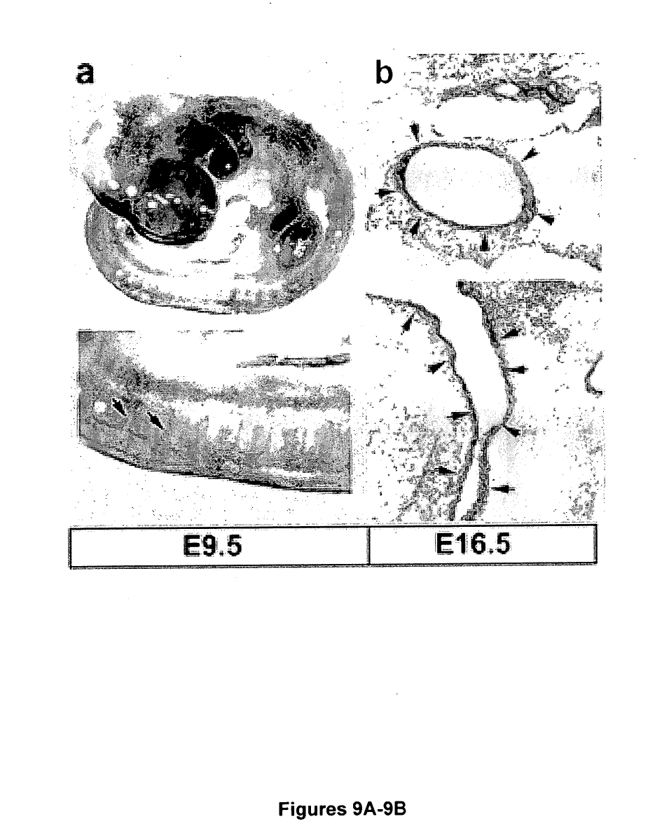

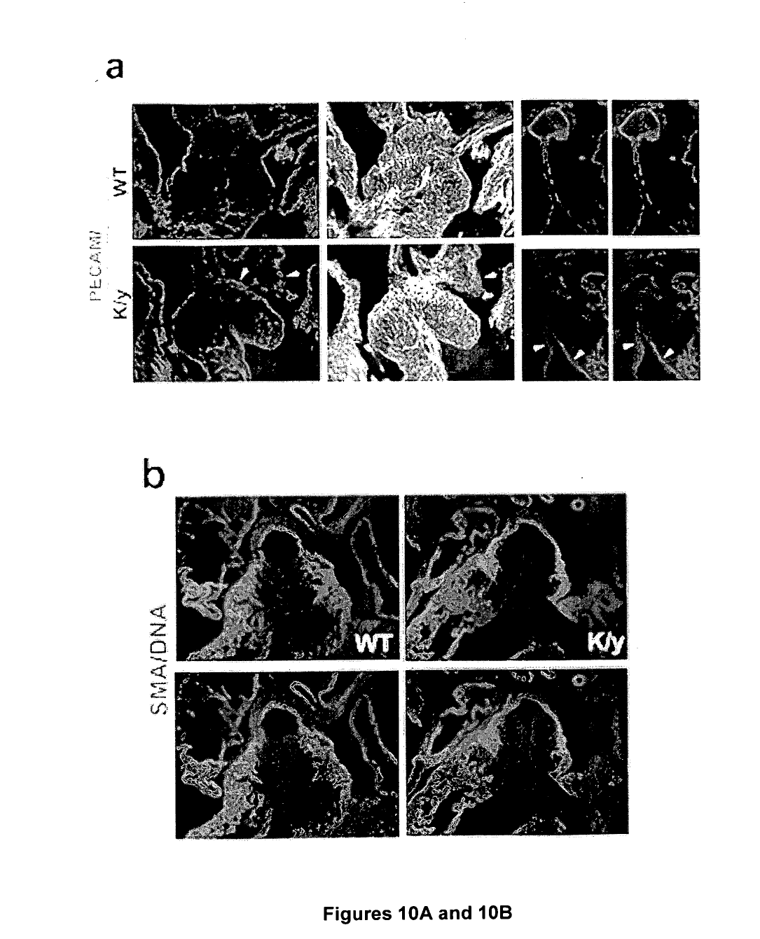

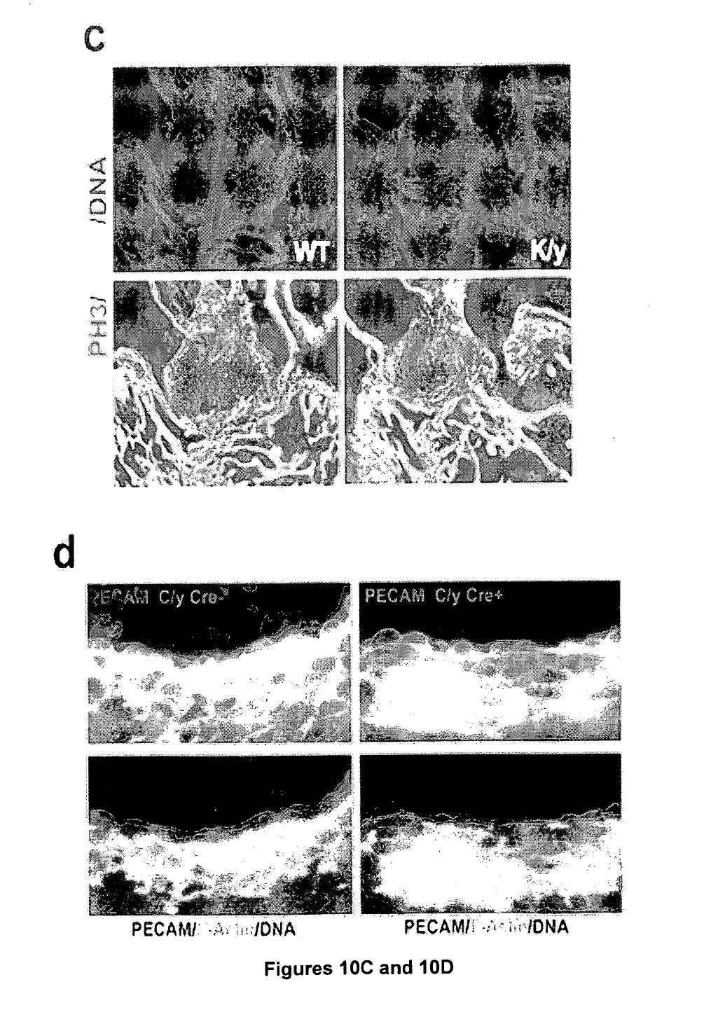

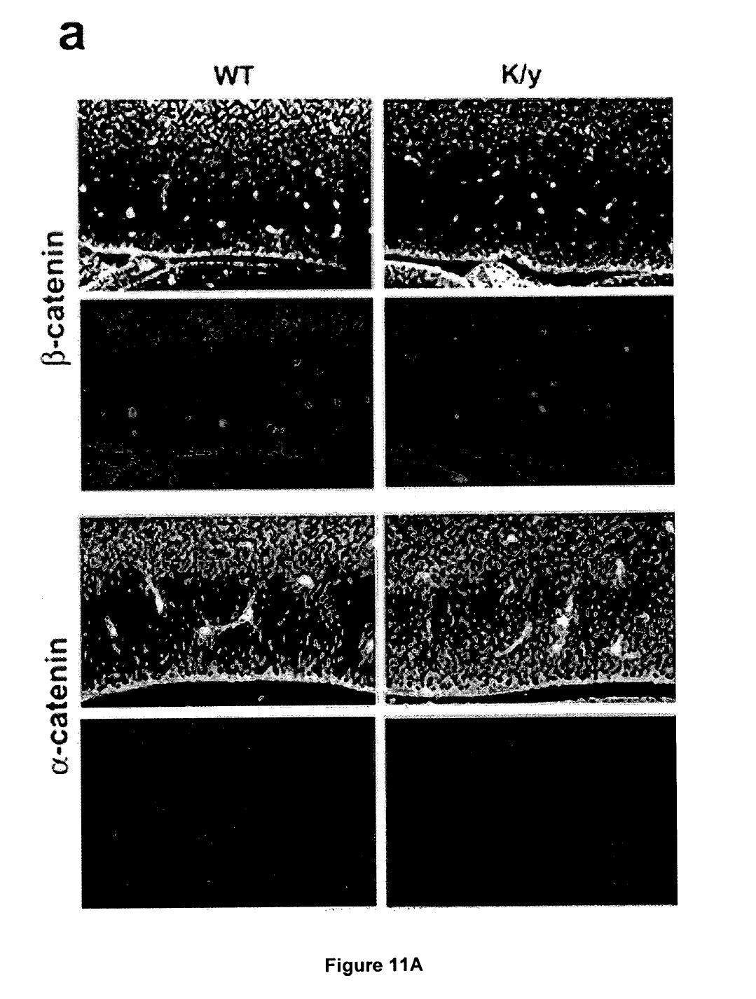

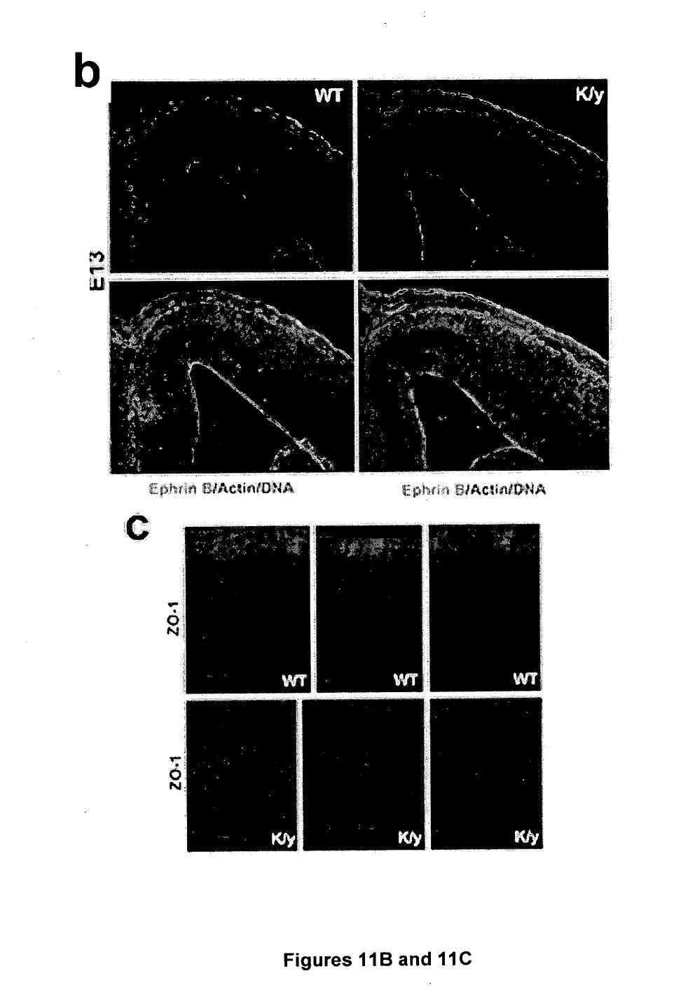

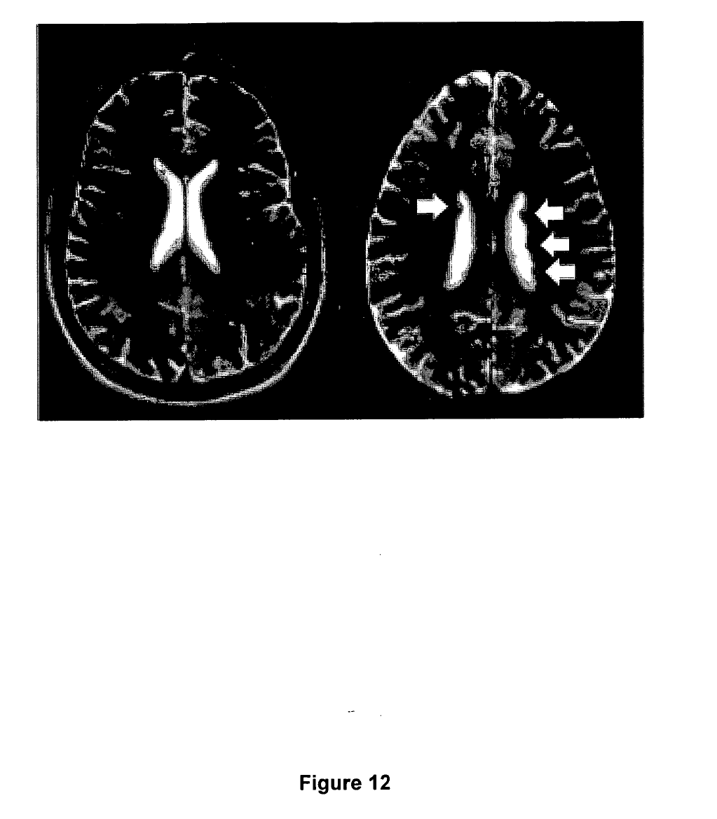

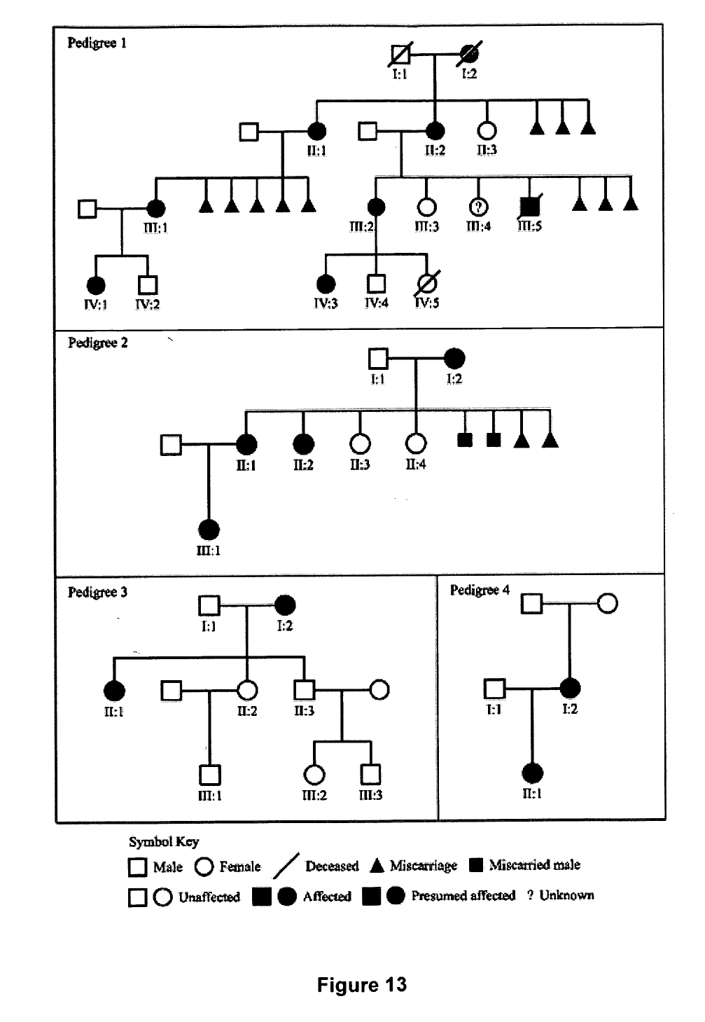

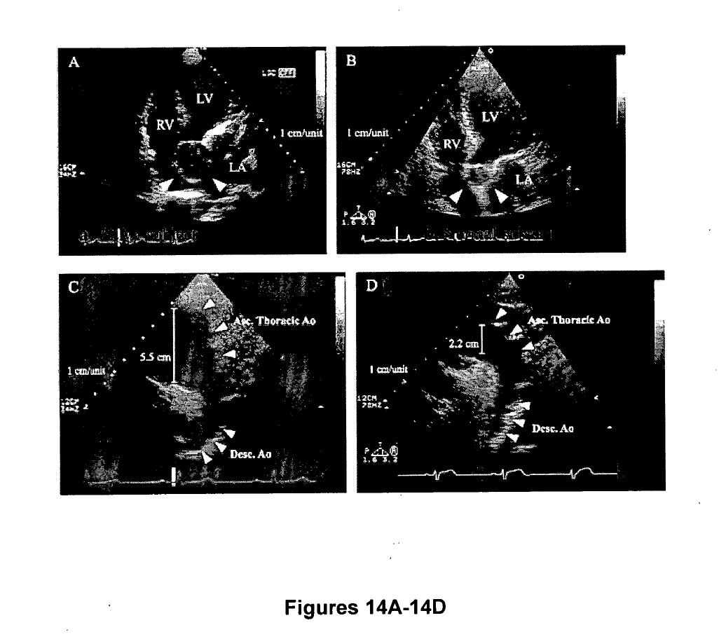

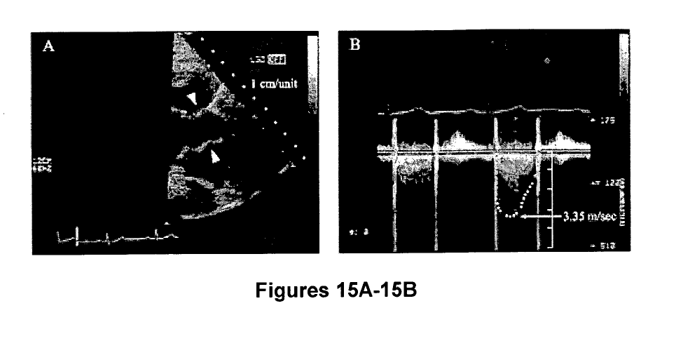

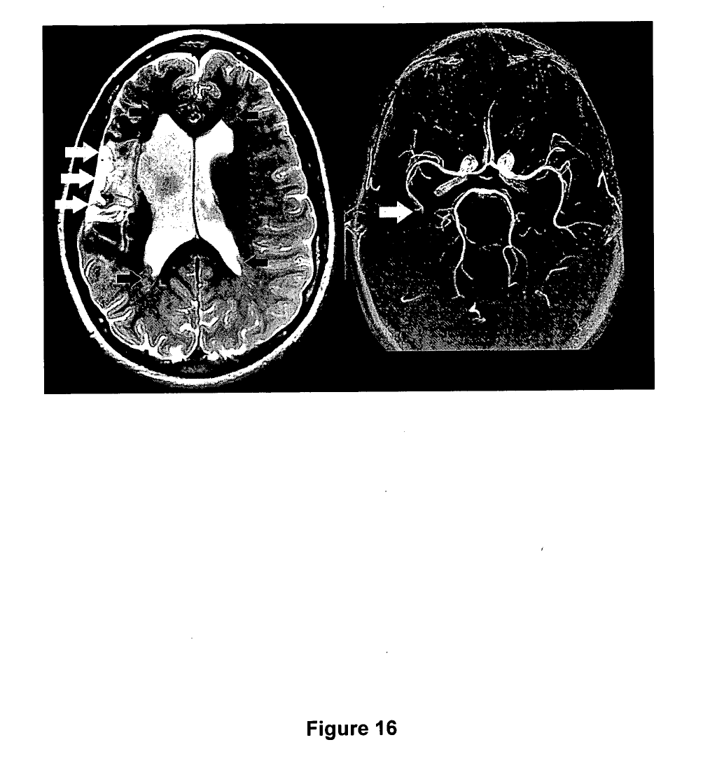

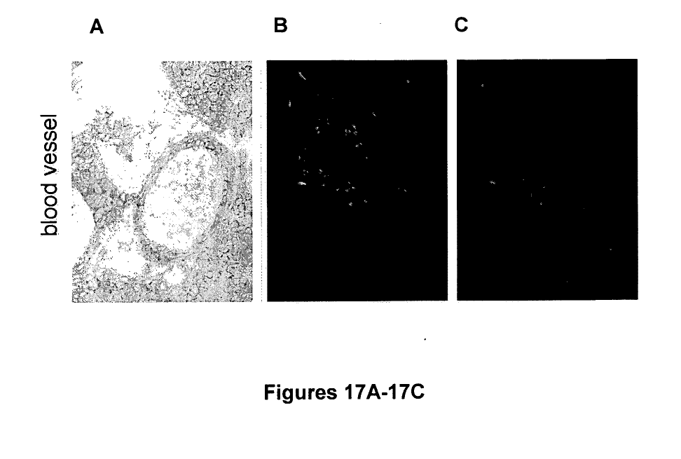

Note the widespread hemorrhage, the abundant dilated blood vessels, and edema in the Flna null (K/y) embryo. FIG. 2B shows a whole-mount immunostaining of E10.5 mouse embryos with a PECAM antibody shows marked disorganization of vasculature in the Flna null mutants (K/y). The red arrow indicates intersomitic vessels and the black arrow indicates somites. The boundaries between somites and intersomitic vessels are missing in the Flna null mutant. FIG. 2C shows immunostaining of cross-sections of spinal cord using an antibody to PECAM (in red) at E12. Note the more abundant and exuberant blood vessels in FlnA mutant sections. Sections are also co-stained with FITC conjugated Phalloidin (for F-actin in green) and Hoechst (in blue).

FIG. 2D shows immunostaining of E1 4 cerebral cortical ventricular epithelial layers with antisera to blood vessel basement membrane protein Nidogen (in red). Note that the blood vessels in the Flna deficient brain are dilated and do not appear to form normal capillaries. However, vessels show intact basement membranes.

FIGS. 3A-3C show cardiac morphogenesis defects caused by Flna deficiency. FIG. 3A shows in situ hybridization analysis of Flna expression on sections of E12.5 mouse embryos. Strong expression of Flna is observed in the endocardial cushion (pink arrows); outflow tract (red arrows) and developing cardiac valves (yellow arrows). FIG. 3B shows whole-mount image of Flna null hearts (K/y) and their wild type littermate at E13.5, showing a single outflow tract (PTA, indicated by arrows) in the K/y embryos. On closer examination, K/y heart displayed persistent truncus arteriosus (PTA) and interrupted aortic arch type B, with an interruption between the left internal carotid artery and left subclavian artery (asterisk). Wild-type hearts show normal aortic arch (arrow head). FIG. 3C shows H&E stained coronal sections of Flna mutant heart (K/y) and of wildtype littermates (WT) at E13.5. All Flna mutant embryos displayed incomplete septation of the ventricles (VSD, arrows) and outflow tracts (PTA, arrow head). A single ventricle is seen (middle right), and combined atrial septal defect-ventricular septal defect is seen (lower right).

FIGS. 4A-4D shows migration independent neural crest defect in cardiac morphogenesis. FIG. 4A shows that Flna conditional knockout with Wntl-Cre transgenic allele caused lethality within the first 24 hours after birth. The skin of the mutant male pups appeared blue, indicating hypoxemia. FIG. 4B shows whole-mount heart images of Flna Wntl-cre mutant males (C/y Cre+) and their wild type or heterozygous female littler mates. C/y Cre+ animals display persistent truncus arteriosus (PTA) and interrupted aortic arch type B, with an interruption between the left internal carotid artery and left subclavian Artery (asterisk). The wild-type hearts show normal aortic arch patterning (asterisk).

FIG. 4C shows pathological analysis (H&E stain) demonstrated that the outflow tract of male Wntl-Cre conditional Flna knockout mice (c/y Cre+) failed to septate, resulting in persistent truncus arteriosus (PTA, arrows).

FIG. 4D shows fate mapping study of neural crest derived cells using ROZA26LacZ Cre reporter, in which the Flna mutant cells were visualized by the co-expression of β-galactosidase. At E12.5, E14.5, E16.5 and P0, Flna deficient cells are distributed normally in all neural crest derived tissues including in cardiac neural crest cells in cardiac outflow tract and endocardial cushion (indicated by red arrows)

FIGS. 5A-5C show normal F-actin structure, motility, and locomotion of Flna null cells. FIG. 5A shows immunofluorescence staining of mouse embryo fibroblasts (MEFs) isolated from Flna null embryos and their littermates at E12.5. Cells were stained with a monoclonal antibody to the focal adhesion protein Vinculin (in green), co-stained with Rhodamine conjugated Phalloidin (in red), and Hoechst (in blue). FIG. 5B shows immunofluorescence staining of Flna null neurons. Flna null neurons were derived by differentiation of multiple lines of Flna null ES cells using 0.5 μM of retinoic acid or from cortical neural progenitor cells. Cells were stained with a monoclonal antibody to neuronal microtubules (Tuj1) to visualize neuronal axons, as well as with Rhodamine conjugated Phalloidin to visualize F-actin in the neuronal growth cone. Cytoskeletal structures in Flna null neurons are essentially indistinguishable from those of the wild type neurons. FIG. 5C shows Flna deficient endothelial cells isolated from E10 embryos and identified by immunostaining with PECAM antibody (in red). F- actin intensity and structure visualized with FITC-conjugated Phalloidin are similar in the Flna null and wild type cells.

FIGS. 6A-6D show defective AJs in Flna deficient vascular endothelial cells. FIGS. 6A-6B show immunostaining with antibodies to

PECAM and VE-Cadherin, which indicates that blood vessel endothelial cells are abnormally organized; and the intensity and distribution of VE-Cadherin are altered in the Flna null embryos at E12.5. Sections were co-stained with FITC-conjugated Phalloidin (for F-actin in green) and Hoechst (for nucleus DNA in blue). Arrows indicate disorganized and atrophic endothelial cells. FIG. 6C shows transmission electron photomicrographs of capillary endothelial cells from Flna null embryos (K/y) and their wild type littermates (WT). Arrows indicate AJs between neighboring endothelial cells. Note that the AJ in the K/y endothelial cells are malformed with reduced electron density. Magnification: 2000× and 5000×, respectively. FIG. 6D shows defective endothelial cell organization in the Flna null hearts. Sections of developing hearts from Flna null embryos and wild type littermates at E12.5 show endothelial cells labeled with a monoclonal antibody to NFATc1 (in red). White arrows indicate endothelial cells in clusters or multiple layers; green arrows indicate regions with absent or defective endothelial lining. Sections were also stained with Hoechst in blue.

FIGS. 7A-7D show brain developmental defects of Flna null mice. FIG. 7A shows that immunostaining with a monoclonal antibody to Flna indicates that Flna is concentrated at the apical surface of neuroepithelial cells in developing cerebral cortex. FIG. 7B shows that immunostaining with VE-Cadherin antibody (in red) indicated its absence from the apical surface of the epithelial cells in cerebral cortex of Flna null embryos at E12.5. Sections were also co-stained with FITC-conjugated Phalloidin (in green) and Hoechst (in blue). FIG. 7C shows Histological analysis of Flna null mutant mice at E14.5- E15 indicating that the mutant brain was smaller, with reduced thickness in cortical plate, and with many dilated blood vessels. FIG. 7D shows immunostaining with DCX antibody, a marker for newly generated cerebral cortical neurons, indicates that postmitotic neurons are in the intermediate zone and cortical plate in Flna null and wild type brain, with no gross accumulation of neurons in the cortical ventricular germinal zone in Flna null mutant.

FIG. 8 is the polypeptide sequence of human (SEQ ID NO:1) Filamin A.

FIGS. 9A and 9B show expression of Flna in developing blood vessels. FIG. 9A shows that whole-mount in situ hybridization of E9.5 mouse embryo with a Flna probe shows strong Flna mRNA expression in the intersomitic blood vessels (arrows). FIG. 9B shows in situ hybridization of sections of E16.5 embryos, showing strong expression of Flna in vascular endothelial cells (arrows).

FIGS. 10A-10D show additional analysis of Flna K/y and Flna C/y; Wntl-Cre mutants. FIG. 10A shows PECAM immunostaining (in red) of the developing endocardial cushion and outflow track at E12.5, showing abnormal organization of the endothelial cells. Regions with multiple layers of endothelial cells are indicated by arrows. Sections were also stained with FITC-conjugated Phalloidin (in green); cell nuclei were stained in blue by Hoechst. FIG. 10B shows immunostaining with a monoclonal antibody to the smooth muscle actin (αSMA, in red), a marker for cardiac mesenchymal cells, which showed normal intensity and distribution in the Flna null endocardial cushion, suggesting grossly normal abundance and organization of mesenchymal cells in the mutant. FIG. 10C shows the endocardial cushion tissue of Flna null mutant also showed normal cell proliferation profile judged by similar immunostaining of phospho-HistoneH3 (in red) in mitotic cells in Flna null and wild type embryos. The sections were also stained with FITC-conjugated Phalloidin, which shows normal structure and intensity of F-actin in the mutant. FIG. 10D shows that, although conditional knockout of Flna in the neural crest cells resulted in septation failure of the cardiac outflow tract, no significant alteration in the great vessel endothelial cells was observed by immunostaining these cells with PECAM (in red). The sections were also stained in green for F-actin and in blue for nuclei.

FIGS. 11A-11C show the alteration in apical localization of VE-Cadherin by Flna mutation is relatively specific. In contrast to in the severe reduction of VE-Cadherin at the apical surface of the cortical neural epithelial cells in Flna null brain (FIG. 7), the intensity and localization of AJ protein α- and β-catenin (FIG. 11A), the tight junction protein ZO-1 (FIG. 11C), and membrane bound ephrin B2 (FIG. 11B), are not obviously changed by Flna mutation, suggesting that the alteration in VE-Cadherin's apical localization by Flna mutation is relatively specific.

FIG. 12 is a pair of images of a brain MRI from a normal patient and a patient with FLNA mutation. The figures show conventional, spin-echo, T2-weighted axial MRI images from a normal individual (left) and from a patient with a FLNA mutation, showing the characteristic periventricular heterotopia (right). The white arrows highlight the nodular heterotopia that lines the ventricles on both sides, protruding into the lateral ventricles. The nodules have signal characteristics indistinguishable from cortical gray matter, and pathologically contain well-developed neurons that resemble cortical neurons.

FIG. 13 is a schematic diagram showing pedigrees of families with FLNA gene mutations. The disorder is inherited in X-linked dominant fashion in virtually all families, with inheritance from mother to daughter. Pedigree 1 illustrates one of the largest and earliest reported pedigrees and shows mother-daughter transmission of the disorder (Fox et al., Neuron 21:1315-1325, 1998). One male (III:5) with a mutation survived embryonic development, but died neonatally with a PDA and widespread hemorrhage.

Affected females show excess miscarriages suggesting prenatal lethality of most affected males. Pedigree 2 shows the same pattern, with two miscarriages occurring late enough so that the fetuses were recognizably male and presumably affected, though they were not examined. Affected females have a shortage of live male offspring, with most live male offspring being normal because they inherited the mother's unaffected X chromosome (see Pedigree 3).

FIGS. 14A-14D are images showing the Sinus of Valsalva and thoracic aortic aneurysm in a patient with a FLNA mutation. FIGS. 14A and 14B show five chamber views of the heart by transthoracic echocardiography illustrating left ventricular outflow tract and aortic root. In FIG. 14A, the Sinus of the Valsalva aneurysm (highlighted with arrowheads) exhibits extreme dilatation of the aortic root at the level of the sinuses. The aneurysm, involving the noncoronary cusp, measures 3.5 cm×3.2 cm. FIG. 14B depicts normal left ventricular outflow tract and aorta. The Sinus of Valsalva is highlighted with arrowheads. Both images were taken at 16 cm scale. FIG. 14C shows a suprasternal view of a large aneurysm of the thoracic aorta, measuring 5.5 cm in maximal diameter and involving the aortic arch (Asc. Thoracic Ao) arch. The descending aorta (Desc. Ao) is not dilated. FIG. 14D shows a corresponding suprasternal view of the aortic arch in an unaffected individual. The unaffected aortic arch measures 2.2 cm in diameter.

FIGS. 15A and 15B are images showing bicuspid aortic valve with aortic stenosis in a patient with a FLNA mutation. FIG. 15A shows a parasternal long-axis view on transthoracic echocardiography and demonstrates deformed aortic valve leaflets with reduced systolic excursion, suggestive of bicuspid aortic valve. A large congenital bicuspid aortic valve was confirmed on surgical pathological examination. The Sinus of Valsalva aneurysm (see FIGS. 14A-14D) is also seen in this view. FIG. 15B shows Doppler assessment of the abnormal aortic valve of the same patient, demonstrating abnormal acceleration of transaortic velocities to 3.35 m/sec. Peak instantaneous and mean gradients are 45/30 mmHg, indicating moderate stenosis.

FIG. 16 is an MRI image showing a stroke in a patient with an FLNA mutation. The figure shows conventional spin-echo T2-weighted magnetic resonance imaging (MRI) of the brain (left) and three-dimensional time-of-flight (3D TOF) magnetic resonance angiography (MRA) of the head (right) of a patient with periventricular nodular heterotopia and a cerebral infarct. The MRI demonstrates the presence of heterotopic nodules of gray matter lining the lateral ventricles bilaterally (black arrows, left) and an area of cerebral infarction in the posterior right frontal lobe (white arrows, left) within the territory of the middle cerebral artery (MCA). The MRA demonstrates an apparent truncation of a right MCA branch (white arrow, right), suggesting the presence of thromboembolism as the etiology of this cerebral infarction. The patient suffered the stroke at age 19 and the MRI was performed at age 35.

FIGS. 17A-17C are images showing expression of FLNA mRNA and protein in vascular structures. FIG. 17A shows in situ hybridization of a mouse blood vessel demonstrating expression of FLNA1 along the endothelial lining. FIGS. 17B and 17C show Filamin A immunostaining in a blood vessel from 33-week-old human fetus. FIG. 17B shows FLNA1 immunostaining (rhodamine) localizing to the periphery of the endothelial cells (red). FIG. 17C demonstrates endothelial cells forming a continuous lining along the internal lumen of the blood vessel, as visualized by Hoechst nuclear staining. The vessel appears to be an arteriole.

### DETAILED DESCRIPTION

We have discovered that Filamin A is involved in reinforcing cell-cell junctions in blood vessels and that mice lacking Flna expression exhibit defects in cell to cell interactions, which result in developmental defects and thus may be associated with cell-cell junction diseases, including vascular diseases such as atherosclerosis, aortic aneurysm, or any disease described herein. We have confirmed the importance of Filiman A in clinical studies of patients with Flna mutations, showing the high rate of cardiovascular disorders which occur in such patients. Accordingly, Flna can be used as a marker in the diagnosis of such disorders, and Flna can represent a target in the treatment of such disorders. The present invention thus features methods and kits for diagnosing a cell-cell junction related disorder or risk of developing such a disorder, methods for treatment of cell-cell junction related disorders, screening methods for identification of compounds to treat cell-cell junction diseases.

Based on this discovery, we also believe that the risk of developing cell-cell junction related disorders (e.g., atherosclerosis) may be correlated with development of Flna somatic mutations. When such a mutation occurs in a dividing cell such as a vascular endothelial cell, disruption of normal Filamin A function or expression can occur. This disruption can weaken cell-cell junctions. This weakening can result in an increased the risk of developing a cell-cell junction related disorder such as atherosclerosis or any disease described herein. In some cases, the Flna gene may have a mutation in its coding region (e.g., a point mutation, insertion, deletion, or truncation). Coding region mutations may result in decreased activity (e.g., actin-binding activity), increased degradation, or decreased stability. In other cases, the mutation or polymorphism may be in a regulatory region of the gene (e.g., promoter, enhancer, or suppressor region), thereby causing an alteration (e.g., decrease) in expression.

Several lines of evidence are consistent with Flna somatic mutations as a risk factor for cell-cell junction related disorders. The Flna gene is located in the Xq28 region of the X chromosome, which is an area subject to loss and rearrangement. In addition, the higher rate of atherosclerosis in men is consistent with a link to somatic mutations in Flna. As men carry a single copy of the X chromosome, a somatic loss-of-function mutation in Flna would be more damaging in men as compared to women, who carry two copies of the gene.

Our work has shown that Flna null mice die at midgestation with widespread hemorrhage from abnormal vessels, persistent truncus arteriosus (PTA, a severe structural malformation of the cardiac outflow tract), as well as incomplete septation of cardiac ventricles and atria. In addition, conditional knockout of Flna in neural crest cells results in perinatal lethality due to abnormalities of the cardiac outflow tract, but the migration of Flna deficient neural crest cells appears to be normal. Importantly, Flna null vascular endothelial cells display severe defects in cell-cell contact and structure of adherens junctions, whereas F-actin structure and integrity in these cells are not detectably altered.

Thus, these data indicate a novel cell motility-independent function of FLNA in establishing proper cell-cell contact and adherens junctions during the development of the heart, blood vessels, and other organs.

Clinical studies of humans having FLNA mutations strongly support the findings in mice. Described herein is the first systematic analysis of the prevalence and significance of cardiac and vascular disease in patients with FLNA mutations. Even when ascertained primarily by interview, questionnaire, and existing medical records, we find a surprisingly high prevalence of congenital heart disease, premature vascular disease, and catastrophic aortic aneurysm, and/or rupture in patients with FLNA mutations. In many cases, cardiovascular disease is the first presentation of FLNA mutations, because the median age of onset of seizures, which are generally the only neurological manifestation of FLNA mutations, is age 15 (see Table 2). Moreover, because many asymptomatic patients declined cardiovascular imaging, the true prevalence of cardiovascular disease in FLNA subjects is probably higher.

Clinical cardiac manifestations of FLNA mutations are somewhat variable, even within a family. Although more than half of FLNA mutation patients show some type of clinically evident cardiac or vascular disease (despite their median age of just 39 years), any given phenotype is observed in less than 50%. The low penetrance is perhaps due to the fact that most of our patients are females heterozygous for X-linked FLNA mutations, and also because many of the vascular phenotypes—particularly aortic aneurysms or cardiovascular and cerebrovascular disease—require time to develop. For example, two patients presently being followed with asymptomatic aortic aneurysm are still in their mid-teenage years. There was also no obvious clustering of vascular malformations, i.e., there were not specific patients with multiple cardiac defects, with other patients showing no cardiovascular defects. Instead, cardiac defects occurred widely in our patients, and were not obviously correlated with neurological outcome or any specific predicted biochemical features of the FLNA mutation.

Congenital anomalies seen with FLNA mutations generally involved the LV outflow tract, specifically affecting the aortic valve, sinuses of Valsalva, the ductus arteriosus, and the ascending thoracic aorta. The ductus arteriosus appears to form by the progressive septation of the aorto-pulmonary trunk-outflow tract (Vaughan et al., Am J Med Genet 97:304-309, 2000). PDA is generally rare, occurring in 1:2000 births. Few genetic causes of PDA have been identified (Vaughan et al., Am J Med Genet 97:304-309, 2000; Math et al., Proc Natl Acad Sci USA 99:15054-15059, 2002). Therefore, FLNA mutations now represent potentially the best-characterized genetic cause of PDA, particularly when observed in the absence of other outward somatic findings, such as are seen in Char syndrome (Satoda et al., Circulation 99:3036-3042, 1999). Hence, the possibility of FLNA mutations should be considered in children with PDA, particularly if they are associated with other congenital heart defects like bicuspid aortic valve or Sinus of Valsalva aneurysm. Because PH can be detected by brain MRI (or head ultrasound in the neonatal period), brain imaging provides rapid, sensitive ascertainment of FLNA mutations in neonates, since DNA-based testing for FLNA mutations identifies fewer than 30% of sporadic cases (Sheen et al., Hum Mol Genet. 10:1775-1783, 2001). The consistent involvement of FLNA in PDA implies that it might be a common downstream target of other genes that regulate normal ductus arteriosus development and closure.

The high incidence of cardiovascular disease observed in patients with FLNA mutations, and the occasional catastrophic consequences, suggest that patients with FLNA mutations, or with periventricular heterotopia by MRI (which is most commonly caused by FLNA mutations) should undergo cardiovascular imaging regardless of symptoms to rule out the presence of aortic aneurysm. Because the offspring of affected patients have a 50% risk of being affected and can be asymptomatic, we believe these offspring should also undergo brain MRI or DNA testing. Finally, given the large size of the FLNA gene and its location near the telomere of the X chromosome, spontaneous mutations of FLNA may also play an important role in vascular disease, especially in males.

There is a remarkable clustering of congenital heart lesions in FLNA mutations, involving the ventricular septum, the aortic valve, the aortic root from the sinus of Valsalva to the ascending thoracic aorta, and with patent ductus arteriosus, yet virtually sparing the other structures of the heart. Interestingly, neural crest cells are thought to be important in the development of each one of these specific regions of the heart (Waldo et al., Dev Biol 208:307-323, 1999). Therefore, a role for Filamin A on neural crest cell development may be the common pathogenosis for these patients with congenital heart disease involving the LV outflow tract and ascending aorta. Mouse models may help elucidate more clearly the mechanisms of Filamin A in cardiac and vascular development.

### Genetic Factors for Aortic Aneurysms

Although genetic factors have long been suspected to play a major role in aortic aneurysms, only two genes have actually been identified (Hasham et al., Curr Opin Cardiol. 17:677-683, 2002). Mutations in FBNJ, encoding Fibrillin 1, cause Marfan's syndrome and are associated with a high incidence of aortic dissection. Similarly, mutations in COL3A, encoding type 3 collagen, are associated with Ehlers-Danlos syndrome type III (so-called vascular Ehlers-Danlos syndrome) and with a similarly high incidence of aortic aneurysmal dissection (Kontusaari et al., J Clin Invest. 86:1465-1473, 1990). Whereas both of these genes cause a distinctive genetic syndrome associated with outwardly observable physical features, the search for genes associated with “nonsyndromic” aortic aneurysm has been slower. Several genetic loci associated with inherited aneurysm have been defined, but these genes have so far not been definitively identified (Wung et al., J Cardiovasc Nurs. 19:409-416, 2004; Vaughan et al., Circulation. 103:2469-2475, 2001; Hasham et al., Ann Emerg Med. 43:79-82, 2004; Hasham et al., Circulation 107:3184-3190, 2003; Kakko et al., J Thorac Cardiovasc Surg 126:106-113, 2003). As described herein, Filamin A appears to be such a gene.

### Filamin A

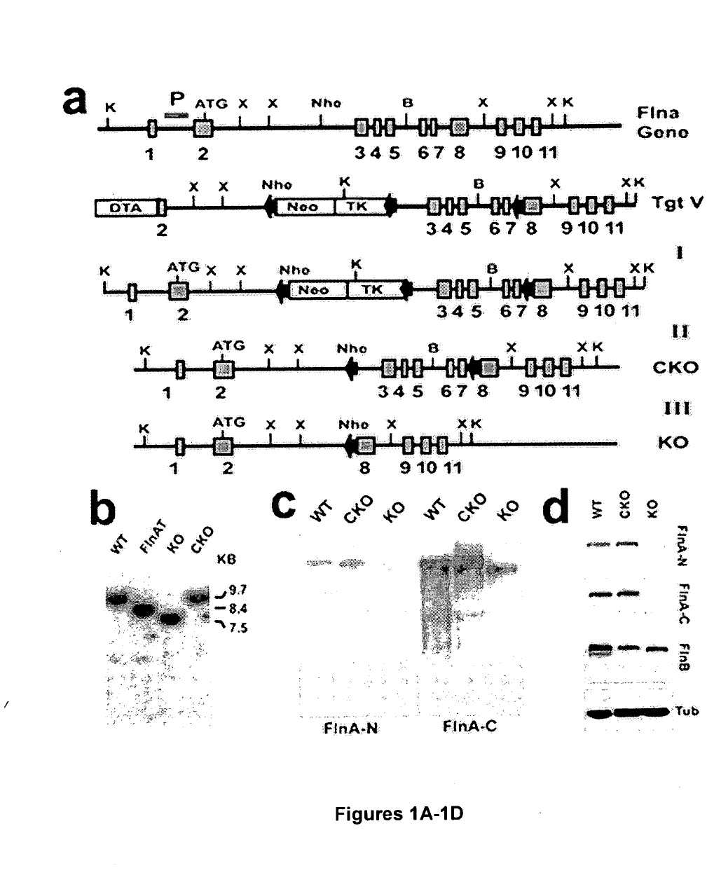

Flna is one of three Filamin isoforms, each of which are essential for normal development in humans (Fox et al., Neuron 21:1315-1325, 1998; Robertson et al., Nat Genet 33:487-491, 2003, Krakow et al., Nat Genet 36:405-410, 2004, Vorgerd et al., Am J Hum Genet 77:297-304, 2005). Flna is essential for cardiac morphogenesis. Each Filamin isoform like has overlapping, yet distinct, functions. Flnb expression overlaps with Flna expression and may provide functional redundancy in some cell types (Takafuta et al., J Biol Chem 273:17531-17538, 1998; Sheen et al., Hum Mol Genet 11:2845-2854, 2002), including mouse ES cells and MEFs used in this study (FIG. 1C and data not shown), which might explain why Flna null mice survive to mid-gestation with a surprisingly normal-appearing actin cytoskeleton. Flnc is specific to muscle cells (Chakarova et al., Hum Genet 107:597-611, 2000). That loss of Flna alone results in severe defects in the heart and blood vessels suggests Flna is essential for cardiovascular morphogenesis.

FLNA encodes an actin-binding protein that is expressed in all non-muscle cells and that links membrane proteins to the actin cytoskeleton. First purified by its actin cross-linking activity (Hartwig et al., J. Biol. Chem. 250:5696-5705, 1975; Wang et al., Proc. Natl. Acad. Sci. USA 72:4483-4486, 1975), Filamin A has been shown to bind a wide variety of cytoplasmic signaling proteins and membrane-bound receptors (Feng et al., Nat Cell Biol 6:1034-1038, 2004). The large number of identified protein-protein interactions of Filamin A suggest similarly widespread functional diversity (Feng et al., Nat Cell Biol 6:1034-1038, 2004). FLNA mutations have been implicated in a form of Ehlers-Danlos syndrome (EDS) (Sheen et al., Neurology 64:254-262, 2005), and certain types of EDS are commonly associated with arterial dissection syndromes. However, most forms of EDS relate to mutations affecting collagen function. FLNA mutations represent a unique, not yet fully characterized cause of EDS that is non-collagen based, and given that FLNA encodes an intracellular, cytoplasmic protein, seems to represent a new mechanism for the pathogenesis of EDS.

### Effects of Flna Loss

Loss of Flna results in aberrant and disorganized vasculature, defective blood vessels with misshapen endothelial cells and abnormal intercellular adherens junctions (AJs), and widespread edema, hemorrhage, and embryonic death. Flna deficient embryos displayed severe cardiac morphogenesis abnormalities involving failure of septation of the ventricles, atria and outflow tracts, leading to persistent truncus arteriosus (PTA), ventricular septal defects (VSD), and atrial septal defects (ASD). The outflow tract defect in Flna null embryos may result partially from abnormalities in cardiac neural crest derived cells, as ablation of Flna from the neural crest cell lineage alone causes defects in outflow track remodeling. Lack of Flna in non-neural crest cells may also contribute to these defects. Surprisingly, given its role as an actin binding protein, the morphogenesis defects associated with Flna mutation are not associated with detectable defects in F-actin stability, cell motility, or cell migration in a variety of cell types. Rather, abnormal cell junctions in the cells of the Flna mutant mice suggest an unexpected role for Flna in establishment and maintenance of AJs.

Despite a large literature linking Flna to cell migration (Stossel et al., Nat Rev Mol Cell Biol 2:138-45, 2001), the most striking cellular defect resulting from loss of Flna is the aberrant AJs. Along with structural defects of AJs, reduced AJ protein VE-Cadherin was seen in vascular endothelial cells, as well as in neuroepithelial cells lining the brain's ventricular system. By regulating endothelial cell growth and contact inhibition, paracellular permeability and homophilic cell-cell adhesion, AJs are essential for the correct organization of new vessels during angiogenesis (Bazzoni et al., Physiol Rev 84:869-901, 2004). Formation and stabilization of AJs require coordinated integration of the cell surface proteins and their intracellular partners that mediate anchorage to the actin cytoskeleton. Flna may stabilize F-actin at the AJ, or may be an adaptor between membrane adhesion/signaling molecules and cytoskeleton anchorage complexes. While Flna deficiency alters the level and localization of VE-Cadherin, changes in other cytoplasmic components of AJs such as a and β-catenins, the tight junction protein ZO-1, or in other membrane molecules such as ephrin B were not observed (FIG. 11). Therefore, Flna may act in parallel with catenins to allow membrane bound AJ molecules to convey signals to the actin cytoskeleton or may act as a molecular switch that regulates actin dynamics at the AJs. Although other membrane-bound molecules may also be altered by Flna mutation, the reduced surface expression of VE-Cadherin in both blood vessels and cortical neuroepithelial cells suggests a role of Flna in AJs.

The role of Flna in adherens junctions links the early, hemorrhagic phenotypes and late vascular phenotypes of Filamin A deficiency. Severe incompetence of vascular endothelial AJs leads to early defects in vascular patterning and integrity. On the other hand, FLNA heterozygous females generally survive embryonic development but are subject to postnatal vascular syndromes that include early onset stroke and aortic dissection (Fox et al., Neuron 21:1315-1325, 1998). As FLNA heterozygous females are mosaics of cells expressing and not expressing Flna due to the X chromosome location of the gene, mosaic dysfunction in Flna null endothelial cells in these patients could explain the variable clinical features.

The role of Flna in heart morphogenesis may involve multiple cell types. Selective removal of Flna in neural crest cells produced malformation of cardiac outflow tracts, suggesting a cell autonomous role of Flna in neural crest cells. Although failure of outflow tract septation is a hallmark of ablation of pre-migratory neural crest cells (Kirby et al., Circ Res 77:211-215, 1995; Stoller et al., Semin Cell Dev Biol 16:704-715, 2005), Flna deficient neural crest cells showed apparently normal migration and targeting into the distal endocardial cushion. Therefore, our data demonstrate a Flna-dependent, post-migratory mechanism that is essential for the differentiation and remodeling of neural crest derivatives after they reach the target tissue.

Flna null hearts are more severely malformed than the Flna Wntl-Cre mutants, suggesting that Flna has critical functions in non-neural crest cells as well as neural crest cells during cardiac development. Flna is expressed highly in endothelial cells and endocardial cushion mesenchymal cells. Flna deficiency appears to affect development of the endocardial cushion, which normally generates the uppermost segment of the interventricular septum. The endocardial cushion arises as swellings of the cardiac jelly that become cellularized as endothelial cells delaminate and undergo an epithelial to mesenchymal transfomation (EMT) (Kim et al., Dev Biol 235:449-466, 2001). EMT involves active steps of cell proliferation; cell identity and shape change from the polygonal epithelial cells to elongated mesenchymal cells; and migration of transformed mesenchymal cells and their reorganization during local tissue remodeling and regression to form cardiac septum and valves (Person et al., Int Rev Cytol 243:287-335, 2005). The observation of disorganized endothelial cells in the developing endocardial cushion and valves in Flna null mutants suggests that Flna plays a role either directly in organizing endothelial cells or in the interaction of endothelial cells with mesenchymal cells. This role in post-migratory and post-transformational remodeling may also rely on Flna mediated cell-cell contact and organization.

Congenital anomalies seen with FLNA mutations generally involved the LV outflow tract, specifically affecting the aortic valve, sinuses of Valsalva, the ductus arteriosus, and the ascending thoracic aorta. The ductus arteriosus appears to form by the progressive septation of the aorto-pulmonary trunk-outflow tract (Vaughan et al., Am J Med Genet 97:304-309, 2000). PDA is generally rare, occurring in 1:2000 births. Very few genetic causes of PDA have been identified (Vaughan et al., Am J Med Genet 97:304-309, 2000; Mani et al., Proc Natl Acad Sci USA 99:15054-15059, 2002), and the molecular genetics are still not well understood. Therefore, FLNA mutations now represent potentially the best-characterized genetic cause of PDA, particularly when observed in the absence of other outward somatic findings, such as are seen in Char syndrome (Satoda et al., Circulation 99:3036-3042, 1999). Hence, the possibility of FLNA mutations should be considered in children with PDA, particularly if they are associated with other congenital heart defects like bicuspid aortic valve or Sinus of Valsalva aneurysm. Because PH can be detected by brain MRI (or head ultrasound in the neonatal period), brain imaging provides rapid, sensitive ascertainment of FLNA mutations in neonates, since DNA-based testing for FLNA mutations identifies fewer than 30% of sporadic cases (Sheen et al., Hum Mol Genet 10:1775-1783, 2001). The consistent involvement of FLNA in PDA implies that it might be a common downstream target of other genes that regulate normal ductus arteriosus development and closure.

Males with FLNA mutations usually die prenatally, though the cause of this early prenatal death has not been definitively determined. Those males with FLNA mutations and PH who survive until birth often die postnatally, and in cases where the cause of death has been determined, it has been catastrophic bleeding (Fox et al., Neuron 21:1315-1325, 1998; Eksioglu et al., Neuron 16:77-87, 1996) or other vascular catastrophe. A similar vascular mechanism may also account for the prenatal lethality of the majority of males. For example, Filamin A protein binding to the glycoprotein 1 b-alpha receptor regulates the platelet activation by von Willebrand's factor (Feng et al., Blood 102:2122-2129, 2003), and so loss of this interaction may also cause defective platelet activation and hence, vascular catastrophe on the basis of a coagulopathy, in addition to potential structural defects of vessels. A male in the study described herein died of aortic rupture in his thirties, consistent with the suggestion that vascular defects from FLNA mutation are more severe in males.

Role of Flna in other Tissues

The finding that Flna is required for the integrity of AJs provides an unexpected explanation for its roles in other tissues, notably brain. The restricted localization of Flna at apical junctions between cerebral cortical neuroepithelial cells provided evidence that defects in cell-cell junctions, rather than requirements in migration, produce the accumulation of neurons at the ventricular surface in human with FLNA mutations. This is supported by the loss of the normally polarized localization of VE-Cadherin in Flna null embryos, and by cases of males with FLNA mutations (Fox et al., Neuron 21:1315-1325, 1998; Sheen et al., Hum Mol Genet 10:1775-1783, 2001; Guerrini et al., Neurology 63:51-56 2004). These males, some of whom were born to mothers with heterozygous FLNA mutations (i.e., are hence not somatic mosaics) demonstrate that many FLNA mutant neurons successfully migrated to the cerebral cortex (Guerrini et al., Neurology 63:51-56 2004). Similarly, analysis of viable Flna^{K/w} mice showed no neuronal heterotopia at one to three months of age. Nonetheless, about 50% of Flna^{K/w} mice died prior to weaning, and variability in the extent of contribution of Flna null cells to various tissues, including the brain, may have contributed to this poor survival. In addition to FLNA mutations, mutations in αSnap and ARFGEF2 also result in periventricular neuronal nodules in mice and humans, respectively (Chae et al., Nat Genet 36:264-270, 2004; Bronson et al., Brain Res Dev Brain Res 54:131-136, 1990; Sheen et al., Brain Dev 26:326-334, 2004). Both αSnap and ARFGEF2 are required for membrane trafficking and polarized targeting of membrane proteins in neuroepithelial cells. As neuroepithelial cell polarity plays an essential role in regulating progenitor cell fate (Chae et al., Nat Genet 36:264-270, 2004; Klezovitch et al., Genes Dev 18:559-571, 2004), defects of cell polarity and adhesion due to Flna deficiency may alter cortical progenitor proliferation and neurogenesis and lead to the ectopic neuronal nodules along the ventricle of PH patients as well as the cytoarchitectural disturbances of cerebral cortex seen in some males with FLNA mutations (Guerrini et al., Neurology 63:51-56 2004).

Finally, PH patients show dyslexia, reduced corpus callosum, and eye movement defects (Chang et al., Neurology 64:799-803, 2005). Although the neurological basis of these defects is unknown, some suggest functions of Filamin A in developing axons. Thus, CNS-specific removal of Flna may allow better understanding the specific mechanism of Flna in developmental control of both neurogenesis and postmitotic neural differentiation.

### FLNA Mutations

Mutations in the X-linked FLNA gene cause a number of human disease syndromes that are often lethal to males before or soon after birth. Heterozygous FLNA mutations in females were first reported to cause an epileptic brain malformation, called periventricular heterotopia (PH), in which a substantial fraction of cerebral cortical neurons fail to complete their normal migration from the periventricular region, adjacent to the lateral ventricles, to the cerebral cortex (Fox et al., Neuron 21:1315-1325, 1998; Eksioglu et al., Neuron 16:77-87, 1996; Kakita et al., Acta Neuropathol (Berl) 104:649-657, 2002; Sheen et al., Hum Mol Genet. 10:1775-1783, 2001) (FIG. 12). Despite the distinctive brain MRI pattern, most patients (75%) have seizures with teenage onset but no other outward signs or symptoms and usually have normal intelligence (Chang et al., Neurology 64:799-803, 2005). Most FLNA mutations appear to be lethal to males prenatally (FIG. 13), resulting in pedigrees with excessive miscarriages and a shortage of male offspring in affected females (Eksioglu et al., Neuron 16:77-87, 1996; Huttenlocher et al., Neurology 44:51-55, 1994). On the other hand, occasional males with FLNA mutations have been reported, and they also seem also to have relatively mild neurological presentations and mutations that may remove some but not all Filamin A function (Sheen et al., Hum Mol Genet. 10:1775-1783, 2001; Moro et al., Neurology 58:916-921, 2002). In contrast to these loss-of-function mutations (whether complete in females or partial in males), specific missense mutations in FLNA cause several other strikingly different and non-overlapping syndromes of skeletal muscle and bone (Otopalatal digital syndrome, Frontometaphyseal dysplasia, and Melnick-Needles syndrome), in which periventricular heterotopia are not observed (Robertson et al., Nat Genet 33:487-491, 2003; Zenker et al., Am J Hum Genet 74(4):731-737, 2004). These specific missense mutations have been postulated to create a “dominant negative” effect by abnormal binding of Filamin A to specific target proteins.

While it was initially surprising that FLNA mutations caused PH, a relatively specific central nervous system (CNS) disorder, a few nonsystematic reports suggested the occasional incidence of non-CNS disorders in patients with PH (Fox et al., Neuron 21:1315-1325, 1998; Kakita et al., Acta Neuropathol (Berl) 104:649-657, 2002; Moro et al., Neurology 58:916-921, 2002). Our initial observation of a few FLNA patients who died from cardiovascular disease at a young age, prompted us to determine systematically the prevalence, range, and severity of cardiovascular defects and events in a larger cohort of patients with confirmed FLNA mutations. As described below, we find that FLNA mutations are associated with frequent disorders of the ascending thoracic aorta and left ventricular outflow tract that warrant careful monitoring and follow-up, because of their potential for catastrophic cardiovascular events.

Results from Studies in Mice

We observed that loss of Flna in mice results in embryonic lethality. Given the lethal effects of complete loss of FLNA function in human males, a conditional knockout strategy was employed in which loxP sites were inserted into introns 2 and 7 of the mouse Flna gene (FIG. 1A). Cre-mediated recombination deletes exons 3 to 7, producing a nonsense mutation with early truncation of the Flna protein at amino acid 121. Targeted ES cell clones were transfected with a plasmid expressing Cre recombinase to generate Flna floxed (or Flna^{c}; Flna cKO) and Flna knockout alleles (Flna^{K}; Flna KO). Genomic modifications were confirmed by Southern and northern analyses (FIGS. 1B and 1C). Western analyses with antisera generated against amino- and carboxyl-terminal portions of FLNA (kindly provided by T. Stossel and J. Hartwig) showed no detectable Flna protein in Flna^{K/y} ES cells or Flna^{K/y} embryos, suggesting the production of a null allele; whereas the Flna^{c/y} ES cells or embryos showed normal level of Flna (FIG. 1D). Two ES cell lines carrying the Flna floxed (Fine) allele were injected into blastocysts to generate conditional Flna knockout mice. These mice were crossed with a β-actin Cre line to generate Flna^{K/y} (Flna null) mice. Breeding of Flna floxed Flna^{c/c} females with β-actin Cre males gave rise to Flna heterozygous knockout females (Flna^{K/w}) but no postnatal male progeny (out of over 100 wild type and Flna^{K/w} analyzed), suggesting that males lacking Flna died before birth.

Some Flna^{K/w} females showed normal development, but about 20% of adult Flna^{K/w} females died within the first 3-4 months and showed many anomalies, including lung edema and emphysema, liver thrombi and necrosis, leukocytosis, and abnormal heart dilation (data not shown), suggesting roles for Flna in vascular integrity after birth. Remaining Flna^{K/w} females were very poor breeders. When Flna^{K/w} female mice were mated with wild type C57BL/6 males, 24% of the female mice at weaning age were Flna^{K/w} (versus 50% expected), confirming embryonic or early postnatal lethality in some Flna^{K/w} heterozygotes (Table 1).

TABLE 1
Null mutation of Flna results in embryonic lethality

| | E10.5-11.5 | E12.5 | E13.5 | E14.5 | E15.5-16.5 | P0-21 |
|---|---|---|---|---|---|---|
| K/y | 17 | 18 | 10 + (1 d) | 8 + (2 d) | 1 + (3 d) | 0 |
| K/w | 19 | 26 | 6 | 13 | 17 | 83 |
| w/w + w/y | 44 | 34 | 28 | 23 | 33 | w/w 261; w/y NC |

Vascular and Cardiac Defects

Flna null mice die of vascular defects. Male Flna^{K/y} mice died at E12.5-E14.5 (Table 1) with widespread vascular defects, including dilated subcutaneous vasculature or hemorrhage (FIG. 2A) and edema, suggesting abnormal vascular permeability. Whole mount in situ hybridization analysis of mouse embryos from E9.5 to E10.5 indicates that Filamin A mRNA is enriched in the limb buds and developing intersomitic vessels (FIG. 9). Blood vessels in Flna null mice were notably coarse and dilated, suggesting failure of fine vascular remodeling (FIGS. 2A and 2C). Whole-mount immunostaining of E10.5 Flna^{K/y} embryos with the blood vessel endothelial marker PECAM (CD31) revealed abnormal patterning of intersomitic blood vessels. Whereas normal intersomitic vessels do not penetrate somites (FIG. 2B), Flna null intersomitic blood vessels extended many aberrant branches and sprouts into somitic tissues (FIG. 2B). Immunohistological examination of E12 embryonic sections with PECAM antibody also displayed disorganized and exuberant growth of the blood vessels in Flna null mutants (FIG. 2C). Nidogen immunostaining showed that the basement membrane of blood vessels in mutant cerebral cortex was intact and continuous at E14 but the vessels were coarser and not remodeled into fine branches (FIG. 2D). Together, these data revealed abnormal angiogenesis in the absence of Flna protein.

Severe cardiac defects were also observed in Flna null mutants. One of the most dramatic effects of Flna deficiency was prominent abnormalities of the cardiovascular outflow tract and aortic arch derivatives, including persistent truncus arteriosus (PTA) and interrupted aortic arch in all (13 of 13) Flna null embryos examined at E13.5 - E14.5. During embryonic development, the pulmonary artery and aorta originate as a common arterial vessel and then septate into two distinct outflow tracts arising from right and left ventricles, respectively. The components of PTA include a single “overriding” outflow tract spanning the two ventricles, an abnormal truncal outflow tract valve, and a membranous ventricular septal defect (VSD) involving the superior segment of the ventricular septum. At E12.5, the Filamin A message is specifically concentrated in the developing endocardial cushion and the cardiac outflow track as well as the inner endothelial layer of major blood vessels (FIG. 3A). Whole-mount heart images, as well as H&E stained sections of Flna deficient heart, displayed a Type I PTA with a single outflow tract overriding the right and left ventricles, an abnormally thickened and malformed outflow tract valve, incomplete septation of the cardiac ventricles with a VSD, and the main pulmonary artery arising from the truncal vessel (FIGS. 3B and 3C). The arch is also interrupted between the left internal carotid artery and the left subclavian artery, resulting in a type B interruption (FIG. 3B). More severe cardiac morphogenesis defects were observed in some Flna null embryos. Some had a single ventricle as seen in serial sections through the heart, and some displayed both atrial septal defect (ASD) and VSD, leading to mixed blood flow between all four heart chambers (FIG. 3C). Together, these observations demonstrated that Flna is essential for cardiac morphogenesis.

Migration-independent neural crest cell defect was also observed. As Flna is thought to be important in cell motility and migration, this cardiac morphogenesis defect may result from a migration defect of neural crest cells, which are essential for cardiac morphogenesis (Creazzo et al., Annu Rev Physiol 60:267-286, 1998; Kirby et al., Circ Res 77:211-215, 1995; Stoller et al., Semin Cell Dev Biol 16:704-715, 2005). Therefore, a Wntl Cre transgenic mouse (Danielian et al., Dev Biol 192:300-309, 1997) was used to excise the Flna floxed allele selectively in the neural crest. Flna^{c/y} Wntl-Cre+ males showed somewhat milder defects than Flna^{K/y} males and survived until birth in

Mendelian ratios, but these males died on the first postnatal day (Table 2) with cyanosis indicating significant hypoxemia (FIG. 4A). All (7 of 7) Flna^{c/y}; Wntl-Cre+ males analyzed showed abnormal cardiac outflow tract structure ranging from PTA to interrupted aortic arch type B, similar to Flna^{K/y} mice, though they were somewhat less severe (FIGS. 4B and 4C). Thus, a cell autonomous defect in neural crest-derived cells is partially responsible for the cardiac outflow and aortic arch patterning defects seen in the Flna null embryos.

TABLE 2
Flna neural crest conditional knockout mice (C/Y Wnt-Cre) die at
birth

| | | | c/y Cre−; | |
|---|---|---|---|---|
| | c/y Cre+ | c/w Cre+ | c/w Cre− | Total/# of litters |
| E17.5-18.5 | 21 | 21 | 48 | 90/7 |
| P0 | 13 (6d) | 26 | 30 | 69/7 |
| P1-1.5 | 0 | 41 | 57 | 98/11 |

To determine whether Flna deficient NC cells were migrating to the developing heart, we introduced the ROSA26-LacZ Cre reporter allele into the Flna^{c/c} mice (Zambrowicz et al., Proc Natl Acad Sci U S A 94:3789-3794, 1997), so that Flna deficient cells are marked by expression of β-galactosidase. Flna deficient neural crest cells appeared to migrate normally as assessed by robust blue staining in all neural crest derived tissues, including the endocardial cushion and cardiac outflow tract (FIGS. 4D-4F), as well as other neural crest derivatives such as cranial vasculature and brachial arch derivatives. The LacZ staining pattern and intensity in Flna^{c/y} Wntl-Cre+ males was indistinguishable from those in the wild type and Flna^{c/w} Wntl-Cre+ female controls; no aberrantly migrated or mis-localized β-galactosidase expressing cells were observed (FIGS. 4D-4F). These data suggest that abnormal migration of neural crest cells does not explain the cardiac defects in Flna mutants, and suggested additional cellular functions of Flna in cardiac neural crest development.

Flna null cells exhibit normal F-actin structure, motility, and locomotion. As Flna is a key actin binding protein and required for maintenance of filamentous actin structures in human melanoma cell lines (Cunningham et al., Science 255:325-327, 1992), we examined F-actin structures and integrity in many cell types from the Flna null mice. Staining saggittal sections of E12.5 to E14.5 embryos with Rhodamine Phalloidin showed no differences in the intensity and structure of F-actin due to Flna deletion (data not shown). Flna deficient mouse embryo fibroblasts (MEFs) showed normal stress fibers and focal adhesions, as revealed by Vinculin immunoreactivity (FIG. 5A). These Flna null MEFs also showed normal motility and dynamic membrane ruffling (data not shown; a video microscopy study of Flna null MEFs demonstrates that the Flna deficient MEFs are motile and exhibit membrane ruffling and locomotion indistinguishable from the wild type MEFs). Flna null mutant ES cells or cortical neural progenitor cells were also able to differentiate into neurons in culture. The Flna null neurons were able to form long axons and normal growth cone structures showed by immunostaining with Tuj-1 antibody and Rhodamine Phalloidin (FIG. 5B). Moreover, in vascular endothelial cells isolated from E10 Flna null embryos, the intensity, architecture and distribution of F-actin were indistinguishable from those in wild type cells (FIG. 5C). These cells also exhibited apparently normal filopodia and other membrane protrusions (FIG. 5C). Collectively, these observations suggest that the cellular defect that underlies Flna's role in blood vessel, heart and neural crest development was not due to the lack of F-actin polymerization or cross linking, but rather due to the loss of other essential functions of Flna.

Flna deficient endothelial cells exhibit cell-cell junction defects. Further analysis of many tissues of the Flna null mice revealed extensive defects in cell-cell junctions, which were particularly well illustrated in vascular endothelial cells. PECAM (CD31) immunostaining of great vessels showed disorganized and discontinuous vascular endothelial cells in Flna null mutants with atrophic, flat or irregular morphology (FIG. 6A, arrows). VE-Cadherin immunoreactivity, normally localized to AJs between vascular endothelial cells, was reduced or absent in Flna null vessels (FIGS. 6B and 6C), suggesting abnormal AJs and weakened endothelial cell-cell contacts. Ultrastructural analysis confirmed that mutant AJs were abnormal, being less electron dense and in many cases unidentifiable with excessive membrane ruffles at sites where AJs normally form (FIG. 6D). The abnormal AJs in Flna null vascular endothelial cells likely cause the hemorrhage and edema in the mutants.

The widespeard cardiac septation defect in Flna null mice may also result from aberrant endothelial cell organization in the remodeling of the endocardial cushion (Kim et al., Dev Biol 235:449-466, 2001). In wild-type embryos at E12.5, endothelial cells, labeled with antiserum to NFATc1, formed a continuous endothelial lining. By contrast, in Flna null mutants, NFATc1 expressing endothelial cells are poorly organized, forming cell clusters or multiple layers in some regions and showing discontinuities or gaps in the lining of both the endocardial cushion and the outflow tract (FIG. 6E). PECAM immunostaining confirmed endothelial disorganization of the outflow tract and endocardial cushion (FIG. 10). However, F-actin, cell proliferation and mesenchymal organization appeared normal in the mutant (FIG. 10). These results are consistent with an abnormality in the organization of endothelial cells in the Flna null mice, suggesting an essential role of Flna in establishing and maintaining cell-cell junctions and organization.

### Neuronal Defects

While defective cell-cell contact and AJs are central to the cardiovascular defects observed in Flna mutants, aberrant AJs in Flna mutant brains also provide a plausible explanation for the heterotopic neurons that line the ventricle in humans with FLNA mutations. Flna protein showed a polarized localization at the apical ventricular surface, where cortical neuroepithelial cells are connected by AJs (Chenn et al., Mol Cell Neurosci 11:183-193, 1998) (FIG. 7A), analogous to its localization in vascular endothelial cells. Although α, β-catenin, zonula occludens-1(ZO-1) and F-actin were normally distributed (FIGS. 7B and 11), VE-Cadherin (Cadherin 5) (Lampugnani et al., J Cell Biol 129:203-217, 1995) lost its normal apical localization in Flna null progenitors (FIG. 7B). Though analysis of Flna null males was limited due to their early lethality, a few surviving E14.5 mutants were examined. Although the architecture of mutant brains was grossly normal, they were smaller than wild type counterparts with a thinner cortical plate, though it was normally positioned (FIG. 7C). Flna deficient neurons were not arrested in the ventricular zone, and at least some neurons in Flna null mutants migrated to the cortical plate by E14.5, and no heterotopic neurons were observed (FIG. 7D). The lack of migratory arrest, together with the aberrant VE-Cadherin localization in Flna null neural epithelial cells, suggests that loss of adherens junctions in the brain may create the periventricular nodules due to architectural disruption of the ventricular lining (Tullio et al., J Comp Neurol 433:62-74, 2001; Zhang et al., (1995) Neurotoxicol Teratol 17:297-311, 1995; Chae et al., Nat Genet 36:264-270, 2004).

### Methods

The following methods were used to generate the results using mice described herein.

### Flna Mutant Mice

Genomic DNA spanning more than 20 kb of mouse Flna gene was obtained by screening a lambda Z70 library. A 2.3 kb fragment containing exon 2 and intron 2 and an 8 kb fragment containing exons 3 through 11 were subcloned into a loxP targeting vector, flanking a Neo-TK cassette (Feng et al., Neuron 44:279-293, 2004). A third loxP site was inserted into intron 7. Strain 129SvEv J1 ES cells (kindly provided by Dr. Arlene Sharp, Brigham & Women's Hospital, Boston, Mass.) were transfected with Notl-linearized targeting vector and selected in 200 μg/ml G418 and G418-resistant colonies were analyzed by Southern blotting. The presence of the loxP site in intron 7 was confirmed by sequencing PCR products using primers flanking the insertion. Positive clones were karyotyped and two clones with >95% diploid chromosomes were transfected with a Cre recombinase plasmid; selected in 2 gancyclovir; and screened for deletion of the Neo-TK cassette alone, or with exon 3-7 of Flna. 2 Flna floxed clones were injected into C57BL/6 blastocysts. Germ-line males were crossed with wild type C57BL/6 females to yield heterozygous Flna floxed females, which were further crossed with β-actin Cre males to generate Flna null heterozygotes. These mice were crossed with wild type C57BL/6 males to remove the β-actin Cre. The Flna heterozygous mutant mice were maintained by breeding with wild type C57BL/6 males. One out of 4 offspring is predicted to be a Flna null male.

Histological and Immunohistological Analysis

Timed embryos (E10 to E17) were collected, genotyped, and fixed with 4% paraformaldehyde. Paraffin sections were stained with H&E. Frozen sections were used for immunohistological analysis with antibodies to: PECAM, VE-Cadherin, NFATc1 (BD Pharmingen), Nidogen (Calbiochem), DCX (Gleeson et al., Neuron 23:257-271, 1999), αSMA (Sigma), phospho Histone H3(Upstate), Flnb and Flna (Takafuta et al., J Biol Chem 273:17531-17538, 1998; Nakamura et al., Blood 107:1925-1932, 2006). At least 3 mutant and 3 wild type littermates were analyzed and representative results are shown. In situ hybridization was performed as described (Sheen et al., Hum Mol Genet 11:2845-2854, 2002). Staining of E10.5 embryos with anti-PECAM/CD31 monoclonal antibody MEC 13.3 (BD Pharmingen) was performed as described (Schlaeger et al., Development 121:1089-1098, 1995).

Cell Culture and Immunostaining

Primary mouse embryo fibroblast (MEF) lines were isolated from E12-E13 mouse embryos and immortalized (Todaro et al., J Cell Biol 17:299-313, 1963). Most analyses were performed using cells from early passages (before P5). Primary vascular endothelial cells from E10 mouse embryos were digested with 0.25% Trypsin at 37° C. for 15 min, then plated on culture dish coated with ECM gel (Sigma), and cultured overnight in FAM's F12K with 10% FBS, 0.1 mg/ml heparin and 5 μg/ml endothelial cell growth supplement (Sigma) and stained as described (Fox et al., Neuron 21:1315-1325, 1998). To generate Flna null neurons, neural progenitors isolated from E13 cerebral cortex were initially cultured as neurospheres in DMEM/F12 with N2 (Invitrogen), 20 ng/ml EGF, 10 ng/ml bFGF (Promega), and 40 mg/ml Heparin. To differentiate progenitors into neurons, neurospheres were washed twice with DMEM/F 12, triturated to break up aggregates, plated on glass cover slips that were pretreated with ECM gel (Sigma), and cultured in DMEM F12 with B27 (Invitrogen).

### Human Clinical Studies

We reviewed and ascertained the cardiac and vascular histories of 36 patients with PH (31 women, 5 men) with confirmed mutations in the FLNA gene. 25% (9/36) of patients with FLNA mutations presented with congenital patent ductus arteriosus (PDA), requiring surgical correction in 8/9, and associated with neonatal demise in 1/9. 6/36 (17%) patients developed ascending thoracic aortic aneurysms at young ages (range: 16-50 years), requiring surgical correction in two and resulting in lethal rupture in the other two before age 41. Congenital valvular heart disease was present in 6/36 (17%) patients. Findings consistent with Ehlers-Danlos syndrome (3/36) and premature strokes (4/36) were also seen.

Whereas neurological signs, especially seizures, are the most common manifestations of FLNA mutations, long-term survival of these patients is more related to cardiovascular disease, suggesting that patients with PH require careful cardiovascular investigation and monitoring. Therefore, FLNA mutations can be considered a cause of congenital PDA requiring surgical correction, or ascending thoracic aortic aneurysm involving the Sinus of Valsalva.

We studied 36 patients (31 women, 5 men) who had genetically confirmed abnormalities of the FLNA gene and sufficiently complete medical interviews and/or medical records to determine the presence or absence of cardiovascular disease (Table 3, Table 4). The surviving patients (n=29) ranged in age from 3 to 63 years (median 39 years), while those patients who were deceased (n=5) died anywhere from 8 days of age to 82 years of age (median 46 years).

TABLE 3
Summary of FLNA gene mutations. The nature of the DNA change,
as well as consequences on mRNA and protein structure, are provided.

| | | | Number | |
|---|---|---|---|---|
| Identifier | FLNA Mutation | mRNA/Protein Change | Affected | Reference |
| FAMILIES | | | | |
| Pedigree 1 | c. 544C > T (exon 3) | p.Q182X | 6 (7)* | ^{a} |
| Pedigree 2 | c. 988-2A > G (exon 7) | Abnormal splice acceptor | 4 | ^{e} |
| Pedigree 3 | c. 6724C > T (exon 41) | p. R2242X | 2 | Herein |
| Pedigree 4 | c. 720 + 2T > C (exon 4) | Abnormal splice donor | 2 | ^{a} |
| Pedigree 5 | c. 245A > T (exon 2) | p.E82V | 3 | ^{b} |
| Pedigree 6 | c. 7627_7634delTGTGCCCC | p. C2542_P 2544 deletion, | 3 | ^{b} |
| | (exon 47) | frameshift, and premature | | |
| | | termination | | |
| Pedigree 7 | c. 446C > T (exon3) | p. S149P | 2 | ^{f} |
| Pedigree 8 | c. 304A > G (exon 2) | p. M102V | 2 | ^{f} |
| Pedigree 9 | c. 1692-2 A > G (exon 12) | Abnormal splice acceptor | 1 | ^{f} |
| SPORADIC FEMALES | | | | |
| Female 1 | c. 287_291delGGCCC (exon 2) | p. R96_P97 deletion, | 1 | ^{a} |
| | | frame shift and premature | | |
| | | termination | | |
| Female 2 | c. 623-3C > G (exon 4) | Abnormal splice acceptor | 1 | ^{a} |
| Female 3 | c. 193 + 1 G > A (exon 2) | Abnormal splice donor | 1 | ^{a} |
| Female 4 | c. 1582G > A (exon 11) | p.V528M | 1 | ^{g} |
| Female 5 | c. 2022 + 1G > A (intron 13) | Abnormal splice donor | 1 | ^{h} |
| Female 6 | c. 4147delG (exon 25) | frameshift mutation and | 1 | ^{d} |
| | | premature protein termination | | |
| Female 7 | c. 2762delG (exon 19) | frameshift mutation and | 1 | ^{d} |
| | | premature protein termination | | |
| Female 8 | c. 116C > G (exon 2) | p.A39G | 1 | ^{d} |
| Female 9 | c. 688C > T | p. C230X | 1 | ^{c} |
| Female 10 | c. 5290G > A | p. A1764T | 1 | ^{c} |
| SPORADIC MALES | | | | |
| Male 1 | c. 6915C > G | p.Y2305X | 1 | ^{c} |

^{a}Fox et al., Neuron 21: 1315-1325, 1998
^{b}Moro et al., Neurology 58: 916-921, 2002
^{c}Sheen et al., Hum Mol Genet 10: 1775-1783, 2001
^{d}Sheen et al., Neurology 64: 254-262, 2005
^{e}Poussaint et al., Pediatr Radiol 30: 748-755, 2000
^{f}Guerrini et al., Neurology 63(1): 51-56, 2004
^{g}Kakita et al., Acta Neuropathol (Berl) 104: 649-657, 2002
^{h}Lange et al., Neurology 62: 151-152, 2004

TABLE 4
Cardiac and other clinical abnormalities associated with FLNA
mutations.

| Abnormality | Number | % Total |
|---|---|---|
| FLNA1 SUBJECTS (n = 36) | | |
| Women | 31 | 86% |
| Deceased | 5 | 14% |
| CVD-related deaths | 3 | |
| Congenital heart disease | | |
| Patent ductus arteriosus | 9 | 25% |
| Surgical correction | 7 | |
| Died at birth | 1 | |
| Aortic aneurysm | 6 | 17% |
| Surgical correction | 2 | |
| Lethal rupture | 2 | |
| Unknown | 2 | |
| Valvular disease | 6 | 17% |
| Aortic valve disease | 4 | |
| Mitral valve disease | 2 | |
| Other congenital defects | 2 | 6% |
| VSD | 2 | |
| ASD | 1 | |
| Vascular disease | | |
| TIA/CVA before 50 yrs old | 4 | 11% |
| Median age (years) | 30 | — |
| % TIA/CVA before 20 yrs old | 50% | — |
| Coronary artery disease | 1 | 3% |
| before 50 yrs old | | |
| Ehlers Danlos Syndrome | 3 | 8% |
| Vein abnormalities | 2 | 6% |
| Easy bruising | 6 | 17% |
| Neurological disease | | |
| Periventricular heterotopia | 36 | 100% |
| Seizures | 22 | 61% |
| Median age of seizure onset (range) | 15 (4-38) | — |

The pedigree structures of 4 families with FLNA mutations are summarized in FIG. 13 in order to illustrate the pattern of genetic inheritance. The largest pedigree contains 7 affected individuals over four generations (6 females and one male who died neonatally), as well as the matriarch of the family (individual 1:2, FIG. 12) who was an obligate carrier of a mutation, but on whom DNA analysis and medical history were not available. This pedigree has been reported before (Fox et al., Neuron 21:1315-1325, 1998; Eksioglu et al., Neuron 16:77-87, 1996), but their cardiovascular history had not been systematically assessed prior to this study. Pedigree 2 consists of four affected individuals from three generations. Pedigrees 3 and 4 each consist of two affected females over two generations. Each of these pedigrees illustrates the “X-linked dominant” inheritance of PH from mother to daughter, with a shortage of surviving sons and an excess of miscarriages, which appear to represent prenatal demise of affected male embryos. Several other smaller pedigrees show the same general pattern of inheritance (Moro et al., Neurology 58:916-921, 2002). In addition, a number of sporadic single cases are included (Table 3). These patients illustrate the high de novo mutation rate of FLNA in PH. 50% or more of FLNA mutation patients have de novo mutations not present in their parents (Sheen et al., Hum Mol Genet. 10:1775-1783, 2001).

Mutation Analysis

Mutations for most of these patients have been previously reported (Fox et al., Neuron 21:1315-1325, 1998; Kakita et al., Acta Neuropathol (Berl) 104:649-657, 2002; Sheen et al., Hum Mol Genet 10:1775-1783, 2001; Moro et al., Neurology 58:916-921, 2002; Sheen et al., Neurology 64:254-262, 2005) but are summarized in Table 3. Newly ascertained individuals in a family with a known mutation were diagnosed as affected if they showed MRI evidence of PH or mutation evidence of a FLNA mutation. No obvious genotype-phenotype correlations were observed, since any given finding was not limited to a given family.

Congenital Patent Ductus Arteriosus

The most common non-CNS defect observed in patients with FLNA mutations is patent ductus arteriosus (PDA), which affected 25% (9/36) of all patients with FLNA mutations. Because not all patients underwent cardiac echocardiography or MRI, and because complete neonatal information was not available on all FLNA mutation patients, this number likely underestimates the prevalence of PDA. PDAs were generally large, requiring surgical or endovascular correction in 8/9 or being associated with neonatal demise in the ninth patient. They usually presented at birth, in full-term infants, though in some cases were not detected until puberty.

Aortic Aneurysm

The second most common disorder associated with FLNA mutations was ascending thoracic aortic aneurysms in 17% of patients (6/36). These generally were detected in young adulthood and required surgical correction in one third (2/6) or resulted in sudden death in another third (2/6). The remaining two patients are being followed medically. FIG. 14 shows a transthoracic echocardiogram of a 36 year old female showing a large thoracic aortic aneurysm involving the sinus of Valsalva and extending to the aortic arch. She underwent successful aortic valve and root replacement. However, during postoperative followup she has shown recurrent dilatation of the aortic root and development of a paravalvular leak. Another patient without a history of hypertension had a mildy dilated thoracic aneurysm (4.2 cm) that was followed by serial echocardiography for ten years before she unexpectedly developed a fatal aortic rupture. That patient also had a small membranous ventricular septal defect.

Valvular Heart Disease

Congenital valvular heart disease was seen in 17% (6/36) of patients and involved only left-sided heart valves. Three patients had congenitally abnormal aortic valves requiring replacement, whereas a fourth had aortic insufficiency. Congenital aortic valve disease was present in 50% (3/6) of patients with thoracic aortic aneurysm. In addition, two other patients had aortic valve disease, one requiring surgery, and the other causing significant aortic insufficiency. FIGS. 15A and 15B, taken from the same patient illustrated in FIG. 14, show an abnormal bicuspid aortic valve resulting in moderate aortic stenosis, in a patient with a sinus of Valsalva aneurysm as well. The preferential involvement of the aortic valve suggests a potential embryonic link to the abnormal ascending thoracic aorta. Two patients with FLNA mutations had mitral valve disease, representing mitral valve prolapse in both cases.

Other Congenital Heart Disease

2/36 (6%) patients had a defect of the left ventricular outflow tract resulting in a ventricular septal defect (VSD). One patient had an atrial septal defect as well.

Vascular Disease

Five patients (14%) with FLNA mutations showed evidence of symptomatic atherosclerotic vascular disease with young onset, presenting either with myocardial infarction or stroke. 4/36 patients (all female) suffered transient ischemic attack (TIA) and/or stroke by the age of 50, with stroke occurring in two patients before 20 years of age (Table 4, FIG. 16). In addition, premature coronary artery disease occurred in one woman <45 years old. Easy bruising was noted in 6 patients (17%). In many cases the easy bruising was associated with flexible joints, suggesting the diagnosis of Ehlers-Danlos syndrome (Sheen et al., Neurology 64:254-262, 2005).

Expression of FLNA mRNA and Protein in Vascular Structures

Although the pattern of expression of mRNA and protein for FLNA has been described in the brain (Sheen et al., Hum Mol Genet. 11:2845-2854, 2002), we tested whether the vascular defects in FLNA mutant subjects reflected expression of Filamin A in blood vessels. We performed in situ hybridization analysis of FlnA mRNA in mouse embryos and noticed abundant hybridization in the walls of all developing vessels (FIG. 17A). Using an antiserum specific to human Filamin A protein, we also examined sections from a 33-week-old human fetus. Abundant Filamin A immunoreactivity characterized the walls of both veins and arterioles at this stage (FIGS. 17B and 17C). The pattern of expression is consistent with Filamin A having potential functions in endothelial integrity in humans.

### Clinical Methods

The following methods were used in the clinical studies described herein.

Patient Methods.

Patient ascertainment. Patients were consecutively enrolled from an existing registry of FLNA patients with periventricular heterotopia. Only those patients with PH that had undergone DNA-based testing that revealed a FLNA mutation were included. Blood samples were obtained by antecubital venipuncture. In order to enroll a consecutive series of patients with FLNA mutations, those patients who had been previously reported as carrying FLNA gene mutations were also included, provided that cardiac history was available or obtainable. In cases where cardiac evaluation had not already been performed, we re-contacted the patients and performed a phone interview about cardiac history and a cardiologist (MHC) reviewed their existing medical records for signs and symptoms of cardiovascular disease.

Questionnaire. We interviewed patients by developing a study questionnaire that queried medical history, and symptoms of cardiac, cranial, and peripheral vascular disease.

Evaluation. We also reviewed patients' medical records and test results. Patients who had been lost to follow-up, and/or for whom no knowledge of cardiovascular history was available (n=1), were excluded. We otherwise included all family members regardless of age. We reviewed existing medical records, test results, cardiac imaging data where available, and where the above were not available, relied on patient self-report through phone interviews; hence, asymptomatic patients who might have harbored silent cardiovascular lesions would not have been detected. We also included information on FLNA mutation cases published by our group and others, provided that information on cardiac disorders was available.

Echocardiography

Transthoracic echocardiograms were obtained on some patients with known FLNA mutations, generally as part of a clinical evaluation. Other patients had it performed during the course of this study.

Immunohistochemistry

In order to determine the expression pattern of FLNA in vascular structures, we performed immunostaining of human tissue (2-month-old) was performed using previously published methods (Sheen et al., Hum Mol Genet. 11:2845-2854, 2002). In brief, paraffin sections (8 microns) were re-hydrated through serial washes in xylene, ethanol, and water. Samples were placed in blocking solution with PBS containing 10% fetal calf serum, 5% horse serum and 5% goat serum, incubated overnight in Filamin A antibody (Novacastra, 1:200 dilution) and processed through standard immunofluorescent conjugated secondary antisera (CY3, Jackson Immunoresearch Laboratories, 1:500 dilution). The sections were visualized using an Olympus fluorescent microscope.

In situ Hybridization

In situ hybridization was performed according to previously described methods (Sheen et al., Hum Mol Genet. 11:2845-2854, 2002; Berger et al., J Comp Neurol. 433:101-114, 2001). The probes were obtained from linearized FlnA and FlnB cDNA templates. The two FlnA oligoprimers contained nucleotides 5′-CAGACCTTAGCCTACTCACAGCC-3′ (SEQ ID NO:2) and 5′-ACTGATCTTCACAGTGAATGGGC-3′ (SEQ ID NO:3).

### Diagnostic Assays

The present invention features assays useful in the diagnosis of a cell-cell junction related disorder such atherosclerosis or diagnosis of an increased risk of developing a cell-cell junction related disorder in a patient, based on the discovery that downregulation of Filamin A results in weak cell-cell junctions and that rupture of vascular endothelial cells can result in increased serum levels of Filamin A. Accordingly, diagnosis of cell-cell junction related disorders can be performed by measuring the level of expression or activity of Filamin A in a sample (e.g., a blood sample) taken from a patient. This level of expression or activity can then be compared to a control sample, for example, a sample taken from a control subject, or to a sample taken from the same patient within ten years (e.g., within 8, 5, 4, 3, 2, or 1 years or within 9, 6, 3, 2, or 1 months). In some cases, a decrease relative to a control sample or decrease over time in the same patient may indicate that the patient is at increased risk of developing or is diagnostic of a cell-cell junction related disorder such as atherosclerosis or any disease described herein. By contrast, a rapid (e.g., within 12 or 24 hours, or within 2, 4, 7, 14, or 28 days) increase in Filamin A levels may be indicative an acute rupture of vascular endothelial cells. This acute increase may likewise be diagnostic of a cell-cell junction related disorder (e.g., atherosclerosis) or indicate and increased risk or propensity to develop a cell-cell junction related disorder.

Analysis of levels of Filamin A mRNA or polypeptides, or activity of the polypeptides, may be used as the basis for screening the biological sample (e.g., a blood sample, tissue sample, or any bodily fluid described herein) taken from the patient. Filamin A nucleic acid and amino acid sequences are available in the art, for example, the nucleic acid amino acid sequences of human Filamin A are provided, for example, in Genbank accession number P21333 (SEQ ID NO:1). Methods for screening mRNA levels include any of those standard in the art, for example, Northern blotting. Methods for screening polypeptide levels may include immunological techniques standard in the art (e.g., Western blot), or may be performed using chromatographic or other protein purification techniques. In another embodiment, the activity (e.g., actin binding activity) of Filamin A may be measured, where an alteration (e.g., decrease) in Filamin A activity is diagnostic of a cell-cell junction related disorder or is indicative of an increased risk in developing a cell-cell junction related disorder.

In other diagnostic assays, the presence of a mutation or polymorphism (e.g., a somatic mutation) in the Flna gene may be used to diagnose a cell-cell junction related disorder or an increased risk of developing such a disorder. Any method for identification of mutants known in the art may be employed. Gene sequencing or hybridization techniques may be employed. Somatic mutations may be identified using PCR (e.g., RT-PCR) and hetero-duplex analysis, for example, as described in Yamada et al., Blood 84:885-892, 1995 hereby incorporated by reference (see, in particular, the Materials and Methods section, pages 885-887). To analyze protein sequences, any art-known method may be used. Techniques such as Western blotting can be used to detect truncated proteins. Peptide sequencing, amino acid analysis, or mass spectroscopy can be used to identify mutant protein sequences (e.g., resulting from point mutations in the gene).

### Diagnostic Kits

The invention also features diagnostic test kits. The diagnostic test kit includes the components required to carry out any of the diagnostic assays described above and instructions for the use of the components to diagnose a cell-cell junction related disorder or the propensity to develop a cell-cell junction related disorder. For example, a diagnostic test kit can include antibodies to Filamin A, and components useful for detecting or evaluating binding between the antibodies and the Filamin A polypeptide. Filamin A antibodies are, for example, commercially available from Bethyl Laboratories (Montgomery, Tex.; Catalog No. A400-034A) and Cell Signaling Technology (Danvers, Mass.; Product No. 4762). For detection, either the antibody or the Filamin A polypeptide is labeled, and either the antibody or the Filamin A polypeptide is substrate-bound, such that the Filamin A polypeptide-antibody interaction can be established by determining the amount of label attached to the substrate following binding between the antibody and the Filamin A polypeptide. In one example, the kit includes a Filamin A binding agent and components for detecting the presence of Filamin A. A conventional ELISA or a sandwich ELSIA is a common, art-known method for detecting antibody-substrate interaction and can be provided with the kit of the invention. Filamin A polypeptides can be detected in virtually any bodily fluid including, but not limited to urine, blood, serum, plasma, saliva, amniotic fluid, or cerebrospinal fluid. A kit that determines an alteration in the level of a Filamin A polypeptide relative to a reference, such as the level present in a normal control, is useful as a diagnostic kit in the methods of the invention. The kit can also include purified proteins to be used as standards in the assay used to detect the level of Filamin A. Desirably, the kit will contain instructions for the use of the kit. In one example, the kit contains instructions for the use of the kit for the diagnosis of a cell-cell junction related disorder (e.g., atherosclerosis or any disease described herein) or the propensity to develop a cell-cell junction related disorder. In another example, the kit contains instructions for the use of the kit to monitor Filamin A levels over time.

In one embodiment of the invention, such a kit includes a solid support (e.g., a membrane or a microtiter plate) coated with a primary agent (e.g., an antibody or protein that recognizes the antigen), standard solutions of purified protein for preparation of a standard curve, a body fluid (e.g., blood or serum) control for quality testing of the analytical run, a secondary agent (e.g., a second antibody reactive with a second epitope in the antigen to be detected or an antibody or protein that recognizes the primary antibody) conjugated to a label or an enzyme such as horse radish peroxidase or otherwise labeled, a substrate solution, a stopping solution, a washing buffer, and an instructions for use.

### Screening Methods to Identify Candidate Therapeutic Compounds

The invention also features screening methods for the identification of compounds that bind to, or modulate expression or activity of Filamin A that may be useful in the treatment of a cell-cell junction related disorder such as atherosclerosis or any disease described herein. In certain embodiments, useful compounds increase the expression or activity of Filamin A.

Screening Assays

Screening assays to identify compounds that alter (e.g., increase) the expression or activity of Filamin A are carried out by standard methods. The screening methods may involve high-throughput techniques. In addition, these screening techniques may be carried out in cultured cells or in organisms such as worms, flies, or yeast. Screening in these organisms may include the use of polynucleotides homologous to human Filamin A (e.g., Drosophila melanogaster or C. elegans homologs).

Any number of methods are available for carrying out such screening assays. According to one approach, candidate compounds are added at varying concentrations to the culture medium of cells expressing a polynucleotide coding for Filamin A. Gene expression is then measured, for example, by standard Northern blot analysis (Ausubel et al., Current Protocols in Molecular Biology, Wiley Interscience, New York, 1997), using any appropriate fragment prepared from the polynucleotide molecule as a hybridization probe. The level of gene expression in the presence of the candidate compound is compared to the level measured in a control culture medium lacking the candidate molecule. A compound which promotes an alteration (e.g., increase) in Filamin A expression is considered useful in the invention; such a molecule may be used, for example, as a therapeutic for a cell-cell junction related disorder (e.g., atherosclerosis).

If desired, the effect of candidate compounds may, in the alternative, be measured at the level of polypeptide production using the same general approach and standard immunological techniques, such as western blotting or immunoprecipitation with an antibody specific for Filamin A. For example, immunoassays may be used to detect or monitor the expression of Filamin A. Polyclonal or monoclonal antibodies which are capable of binding to such a polypeptide may be used in any standard immunoassay format (e.g., western blot or RIA assay) to measure the level of Filamin A. A compound which promotes an increase the expression of Filamin A is considered particularly useful. Again, such a molecule may be used, for example, as a therapeutic for a cell-cell junction related disorder (e.g., atherosclerosis or any disease described herein).

Alternatively, or in addition, candidate compounds may be screened for those which specifically bind to and activate Filamin A. The efficacy of such a candidate compound is dependent upon its ability to interact with the polypeptide. Such an interaction can be readily assayed using any number of standard binding techniques and functional assays (e.g., those described in Ausubel et al., supra). For example, a candidate compound may be tested in vitro for interaction and binding with Filamin A and its ability to modulate (e.g., increase) its activity may be assayed by any standard assays (e.g., those described herein).

In one particular embodiment, a candidate compound that binds to Filamin A may be identified using a chromatography-based technique. For example, recombinant Filamin A may be purified by standard techniques from cells engineered to express Filamin A and may be immobilized on a column. A solution of candidate compounds is then passed through the column, and a compound specific for Filamin A is identified on the basis of its ability to bind to the polypeptide and be immobilized on the column. To isolate the compound, the column is washed to remove non-specifically bound molecules, and the compound of interest is then released from the column and collected. Compounds isolated by this method (or any other appropriate method) may, if desired, be further purified (e.g., by high performance liquid chromatography). Compounds isolated by this approach may also be used, for example, as therapeutics to treat a cell-cell junction related disorder (e.g., atherosclerosis or any disease described herein). Compounds which are identified as binding to Filamin A with an affinity constant less than or equal to 10 mM are considered particularly useful in the invention.

Potential agonists and antagonists include organic molecules, peptides, peptide mimetics, polypeptides, and antibodies that bind to Filamin A or a polynucleotide encoding Filamin A and thereby alter (e.g., increase) its activity.

Polynucleotide sequences coding for Filamin A may also be used in the discovery and development of compounds to treat disorders involving cell-cell junction related disorders (e.g., atherosclerosis or any disease described herein).

Filamin A, upon expression, can be used as a target for the screening of drugs. Additionally, the polynucleotide sequences encoding the amino terminal regions of the encoded polypeptide or Shine-Delgarno or other translation facilitating sequences of the respective mRNA can be used to construct antisense sequences to control the expression of the coding sequence of interest.

The antagonists and agonists of the invention may be employed, for instance, to treat a variety of cell-cell junction related disorders such as atherosclerosis or any disease described herein.

Optionally, compounds identified in any of the above-described assays may be confirmed as useful in delaying or ameliorating cell-cell junction related disorders in either standard tissue culture methods or animal models and, if successful, may be used as therapeutics for treating such disorders.

Small molecules provide useful candidate therapeutics. Preferably, such molecules have a molecular weight below 2,000 daltons, more preferably between 300 and 1,000 daltons, and most preferably between 400 and 700 daltons. It is preferred that these small molecules are organic molecules.

Test Compounds and Extracts

In general, compounds capable of treating a cell-cell junction related disorder (e.g., atherosclerosis or any disease described herein) are identified from large libraries of both natural product or synthetic (or semi-synthetic) extracts or chemical libraries according to methods known in the art. Those skilled in the field of drug discovery and development will understand that the precise source of test extracts or compounds is not critical to the screening procedure(s) of the invention. Accordingly, virtually any number of chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and polynucleotide-based compounds. Synthetic compound libraries are commercially available. Alternatively, libraries of natural compounds in the form of bacterial, fungal, plant, and animal extracts are commercially available. In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their activity in treating cell-cell junction related disorders should be employed whenever possible.

When a crude extract is found to have an activity that alters (e.g., increases) Filamin A expression or activity, or a binding activity, further fractionation of the positive lead extract is necessary to isolate chemical constituents responsible for the observed effect. Thus, the goal of the extraction, fractionation, and purification process is the characterization and identification of a chemical entity within the crude extract having activity that may be useful in treating a cell-cell junction related disorder (e.g., atherosclerosis or any disease described herein). Methods of fractionation and purification of such heterogenous extracts are known in the art. If desired, compounds shown to be useful agents for the treatment of a cell-cell junction related disorder (e.g., atherosclerosis or any disease described herein) are chemically modified according to methods known in the art.

### Treatment of a Cell-Cell Junction Related Disorder

The invention also features methods for treating a cell-cell junction related disorder such as atherosclerosis or any disease described herein by administration of a composition or compound that alters (e.g., increases) expression or activity of Filamin A in a patient (e.g., a human such as an adult human). The compounds used in the treatment of cell-cell junction related disorders may, for example, be compounds identified using the screening methods described herein.

Gene Therapy

Alterations (e.g., increases) in Filamin A expression or activity may be achieved through introduction of gene vectors into a patient. To treat a cell-cell junction related disorder such as atherosclerosis or any disease described herein, Filamin A expression may be altered (e.g., increased), for example, by administering to a subject a vector containing a polynucleotide sequence encoding Filamin A, operably linked to a promoter capable of driving expression in targeted cells (e.g., vascular endothelial cells). In another approach, a polynucleotide sequence encoding a protein that alters (e.g., increases) transcription of the Filamin A gene may be administered to a patient with a cell-cell junction related disorder (e.g., atherosclerosis or any disease described herein) or at increased risk of developing such a disorder. Any standard gene therapy vector and methodology may be employed for such administration. In certain embodiments, the gene vector may be coated onto a stent, which allows for localized delivery of the gene vector the area affected by a cell-cell junction related disorder such as atherosclerosis or any disease described herein.

Formulation of Pharmaceutical Compositions

The administration of any compound described herein or identified using the methods of the invention may be by any suitable means that results in a concentration of the compound that treats a cell-cell junction related disorder (e.g., atherosclerosis or any disease described herein). The compound may be contained in any appropriate amount in any suitable carrier substance, and is generally present in an amount of 1-95% by weight of the total weight of the composition. The composition may be provided in a dosage form that is suitable for the oral, parenteral (e.g., intravenously or intramuscularly), rectal, cutaneous, nasal, vaginal, inhalant, skin (patch), ocular, or intracranial administration route. Thus, the composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, or aerosols. The pharmaceutical compositions may be formulated according to conventional pharmaceutical practice (see, e.g., Remington: The Science and Practice of Pharmacy, 20th edition, 2000, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York).

Pharmaceutical compositions may be formulated to release the active compound immediately upon administration or at any predetermined time or time period after administration. The latter types of compositions are generally known as controlled release formulations, which include (i) formulations that create substantially constant concentrations of the agent(s) of the invention within the body over an extended period of time; (ii) formulations that after a predetermined lag time create substantially constant concentrations of the agents of the invention within the body over an extended period of time; (iii) formulations that sustain the agent(s) action during a predetermined time period by maintaining a relatively constant, effective level of the agent(s) in the body with concomitant minimization of undesirable side effects associated with fluctuations in the plasma level of the agent(s) (sawtooth kinetic pattern); (iv) formulations that localize action of agent(s), e.g., spatial placement of a controlled release composition adjacent to or in the diseased tissue or organ; (v) formulations that achieve convenience of dosing, e.g., administering the composition once per week or once every two weeks; and (vi) formulations that target the action of the agent(s) by using carriers or chemical derivatives to deliver the compound to a particular target cell type. Administration of the compound in the form of a controlled release formulation is especially preferred for compounds having a narrow absorption window in the gastro-intestinal tract or a relatively short biological half-life.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the compound is formulated with appropriate excipients into a pharmaceutical composition that, upon administration, releases the compound in a controlled manner. Examples include single or multiple unit tablet or capsule compositions, oil solutions, suspensions, emulsions, microcapsules, molecular complexes, microspheres, nanoparticles, patches, and liposomes.

Stents

A stent acts as a scaffold to provide structural support for a vessel. A stent, or a small, expandable wire tube, is often used in the treatment of coronary artery disease. During angioplasty, the balloon is placed inside the stent and inflated, and this opens the stent and pushes it into place against the artery wall. The stent is left permanently and often, because the stent is meshlike, the cells lining the blood vessel grow through and around the stent to help secure it. Stents are commonly used in angioplasty to restore and maintain adequate blood flow to the heart and to prevent the artery wall from collapsing or closing again.

In the present invention, a method is featured for treating, preventing onset of, or reducing risk of a cell-cell junction related disorder, using a stent coated with a therapeutically effective amount of composition capable of altering (e.g., increasing) expression or activity of Filamin A. In certain embodiments, the stent is coated with vector containing a polynucleotide sequence coding for Filamin A. The vector may be capable of expression in a vascular endothelial cell.

Stents are coated using standard methods known in the art. Methods for coating stents are generally known and examples can be found in U.S. Pat. Nos. 6,153,252; 6,258,121; and 5,824,048, hereby incorporated by reference. For each of the therapeutic compounds listed in the present application, the amount of therapeutic agent used will be dependent upon the particular drugs employed. Typically, the amount of drug represents about 0.001% to about 70%, more typically about 0.001% to about 60%, most typically about 0.001% to about 45% by weight of the coating.

### Other Embodiments

All publications, patent applications, and patents mentioned in this specification are herein incorporated by reference including U.S. Provisional Application No. 60/872,169, filed Dec. 1, 2006 and 60/872,731, filed Dec. 4, 2006.

Various modifications and variations of the described methods and kits of the invention will be apparent to those skilled in the art without departing from the scope and spirit of the invention. Although the invention has been described in connection with specific desired embodiments, it should be understood that the invention as claimed should not be unduly limited to such specific embodiments. Indeed, various modifications of the described modes for carrying out the invention that are obvious to those skilled in the fields of medicine, immunology, pharmacology, or related fields are intended to be within the scope of the invention.

What is claimed is:

1. A method for diagnosing a cell-cell junction related disorder or an increased propensity thereto in a patient, said method comprising the steps:
(a) obtaining a biological sample from said patient; and
(b) determining the level of Filamin A in said sample, wherein an alteration in the level of Filamin A is diagnostic of a cell-cell junction related disorder or an increased propensity thereto.

2. A method for diagnosing a cell-cell junction related disorder or an increased propensity thereto in a patient, said method comprising the steps:
(a) obtaining a first biological sample from said patient;
(b) determining the level of Filamin A in said first sample;
(c) obtaining a second biological sample from said patient within five years of said first sample; and
(d) determining the level of Filamin A in said second sample, wherein an alternation in the level in said second sample as compared to said first sample is diagnostic of a cell-cell junction related disorder or an increased propensity thereto.

3. The method of claim 2, wherein steps (c) and (d) are repeated at least one additional time.

4. The method of claim 2, wherein said second sample is taken within one year of said first sample.

5-6. (canceled)

7. The method of claim 1, wherein said cell-cell junction related disorder is atherosclerosis.

8. The method of claim 1, wherein said determining steps are performed in conjunction measuring the level of a vascular endothelial cell marker.

9. The method of claim 8, wherein said marker is PECAM or factor VIII.

10. The method of claim 1, wherein said patient is an adult.

11. The method of claim 1, wherein said biological sample is a blood sample.

12. The method of claim 1, wherein said alteration is a decrease.

13-23. (canceled)

24. A method for treating a cell-cell junction related disorder in a patient in need thereof, said method comprising administering a composition that increases the expression or activity of Filamin A in said patient in an amount sufficient to treat said patient.

25. The method of claim 24, wherein said method increases expression or activity of Filamin A in an endothelial cell.

26. The method of claim 25, wherein said endothelial cell is a vascular endothelial cell.

27. The method of claim 24, wherein said composition comprises a polynucleotide encoding Filamin A.

28. The method of claim 27, wherein said polynucleotide is part of a vector.

29. The method of claim 27, wherein said polynucleotide is coated onto a stent.

30. The method of claim 24, wherein said disorder is atherosclerosis.

31. The method of claim 24, wherein said patient is an adult.

32. The method of claim 24, wherein said patient is a human.


SEQUENCE LISTING (3 sequences)

SEQ ID NO: 1 - protein, 2647 aa, Homo sapiens
MSSSHSRAGQSAAGAAPGGGVDTRDAEMPATEKDLAEDAPWKKIQQNTFTRWCNEHLKCVSKRIANLQTDLSDGLRLIALLEVLSQKKMHRKHNQRPTFRQMQLENVSVALEFLDRESIKLVSIDSKAIVDGNLKLILGLIWTLILHYSISMPMWDEEEDEEAKKQTPKQRLLGWIQNKLPQLPITNFSRDWQSGRALGALVDSCAPGLCPDWDSWDASKPVTNAREAMQQADDWLGIPQVITPEEIVDPNVDEHSVMTYLSQFPKAKLKPGAPLRPKLNPKKARAYGPGIEPTGNMVKKRAEFTVETRSAGQGEVLVYVEDPAGHQEEAKVTANNDKNRTFSVWYVPEVTGTHKVTVLFAGQHIAKSPFEVYVDKSQGDASKVTAQGPGLEPSGNIANKTTYFEIFTAGAGTGEVEVVIQDPMGQKGTVEPQLEARGDSTYRCSYQPTMEGVHTVHVTFAGVPIPRSPYTVTVGQACNPSACRAVGRGLQPKGVRVKETADFKVYTKGAGSGELKVTVKGPKGEERVKQKDLGDGVYGFEYYPMVPGTYIVTITWGGQNIGRSPFEVKVGTECGNQKVRAWGPGLEGGVVGKSADFVVEAIGDDVGTLGFSVEGPSQAKIECDDKGDGSCDVRYWPQEAGEYAVHVLCNSEDIRLSPFMADIRDAPQDFHPDRVKARGPGLEKTGVAVNKPAEFTVDAKHGGKAPLRVQVQDNEGCPVEALVKDNGNGTYSCSYVPRKPVKHTAMVSWGGVSIPNSPFRVNVGAGSHPNKVKVYGPGVAKTGLKAHEPTYFTVDCAEAGQGDVSIGIKCAPGVVGPAEADIDFDIIRNDNDTFTVKYTPRGAGSYTIMVLFADQATPTSPIRVKVEPSHDASKVKAEGPGLSRTGVELGKPTHFTVNAKAAGKGKLDVQFSGLTKGDAVRDVDIIDHHDNTYTVKYTPVQQGPVGVNVTYGGDPIPKSPFSVAVSPSLDLSKIKVSGLGEKVDVGKDQEFTVKSKGAGGQGKVASKIVGPSGAAVPCKVEPGLGADNSVVRFLPREEGPYEVEVTYDGVPVPGSPFPLEAVAPTKPSKVKAFGPGLQGGSAGSPARFTIDTKGAGTGGLGLTVEGPCEAQLECLDNGDGTCSVSYVPTEPGDYNINILFADTHIPGSPFKAHVVPCFDASKVKCSGPGLERATAGEVGQFQVDCSSAGSAELTIEICSEAGLPAEVYIQDHGDGTHTITYIPLCPGAYTVTIKYGGQPVPNFPSKLQVEPAVDTSGVQCYGPGIEGQGVFREATTEFSVDARALTQTGGPHVKARVANPSGNLTETYVQDRGDGMYKVEYTPYEEGLHSVDVTYDGSPVPSSPFQVPVTEGCDPSRVRVHGPGIQSGTTNKPNKFTVETRGAGTGGLGLAVEGPSEAKMSCMDNKDGSCSVEYIPYEAGTYSLNVTYGGHQVPGSPFKVPVHDVTDASKVKCSGPGLSPGMVRANLPQSFQVDTSKAGVAPLQVKVQGPKGLVEPVDVVDNADGTQTVNYVPSREGPYSISVLYGDEEVPRSPFKVKVLPTHDASKVKASGPGLNTTGVPASLPVEFTIDAKDAGEGLLAVQITDPEGKPKKTHIQDNHDGTYTVAYVPDVTGRYTILIKYGGDEIPFSPYRVRAVPTGDASKCTVTVSIGGHGLGAGIGPTIQIGEETVITVDTKAAGKGKVTCTVCTPDGSEVDVDVVENEDGTFDIFYTAPQPGKYVICVRFGGEHVPNSPFQVTALAGDQPSVQPPLRSQQLAPQYTYAQGGQQTWAPERPLVGVNGLDVTSLRPFDLVIPFTIKKGEITGEVRMPSGKVAQPTITDNKDGTVTVRYAPSEAGLHEMDIRYDNMHIPGSPLQFYVDYVNCGHVTAYGPGLTHGVVNKPATFTVNTKDAGEGGLSLAIEGPSKAEISCTDNQDGTCSVSYLPVLPGDYSILVKYNEQHVPGSPFTARVTGDDSMRMSHLKVGSAADIPINISETDLSLLTATVVPPSGREEPCLLKRLRNGHVGISFVPKETGEHLVHVKKNGQHVASSPIPVVISQSEIGDASRVRVSGQGLHEGHTFEPAEFIIDTRDAGYGGLSLSIEGPSKVDINTEDLEDGTCRVTYCPTEPGNYIINIKFADQHVPGSPFSVKVTGEGRVKESITRRRRAPSVANVGSHCDLSLKIPEISIQDMTAQVTSPSGKTHEAEIVEGENHTYCIRFVPAEMGTHTVSVKYKGQHVPGSPFQFTVGPLGEGGAHKVRAGGPGLERAEAGVPAEFSIWTREAGAGGLAIAVEGPSKAEISFEDRKDGSCGVAYVVQEPGDYEVSVKFNEEHIPDSPFVVPVASPSGDARRLTVSSLQESGLKVNQPASFAVSLNGAKGAIDAKVHSPSGALEECYVTEIDQDKYAVRFIPRENGVYLIDVKFNGTHIPGSPFKIRVGEPGHGGDPGLVSAYGAGLEGGVTGNPAEFVVNTSNAGAGALSVTIDGPSKVKMDCQECPEGYRVTYTPMAPGSYLISIKYGGPYHIGGSPFKAKVTGPRLVSNHSLHETSSVFVDSLTKATCAPQHGAPGPGPADASKVVAKGLGLSKAYVGQKSSFTVDCSKAGNNMLLVGVHGPRTPCEEILVKHVGSRLYSVSYLLKDKGEYTLVVKWGHEHIPGSPYRVVVP

SEQ ID NO: 2 - DNA, 23 bp, Artificial Sequence
  Description of Artificial Sequence Synthetic construct
cagaccttagcctactcacagcc

SEQ ID NO: 3 - DNA, 23 bp, Artificial Sequence
  Description of Artificial Sequence Synthetic construct
actgatcttcacagtgaatgggc